(19) United States
(12) Patent Application Publication
(10) Pub. No.: US 2020/0376128 A1
(43) Pub. Date: Dec. 3, 2020

(54) MONODISPERSE RESORBABLE POLYESTER POLYMER COMPOSITIONS, SYSTEMS, AND METHODS

(71) Applicant: LUPIN HOLDINGS B.V., Amsterdam (NL)

(72) Inventors: Okke Franssen, Ambt-Delden (NL); Emilie Blazejewski, Bad Bentheim (DE); Evelyn Koekoek, Rotterdam (NL); Jaap Rip, Leiden (NL)

(21) Appl. No.: 16/888,803

(22) Filed: May 31, 2020

Related U.S. Application Data
(60) Provisional application No. 62/856,000, filed on Jun. 1, 2019.

(51) Int. Cl. A61K 47/34; A61K 8/04; A61K 9/06; A61K 8/85

(52) U.S. Cl. CPC A61K 47/34; A61K 8/042; A61K 9/06; A61K 9/0019; A61K 2800/91; A61K 2800/412; A61K 8/85

(57) ABSTRACT

Described herein are novel compositions comprising resorbable polyester polymers, such as poly-L-lactic acid particles, including novel compositions that function as neocollagenic dermal implants and that have unique physical properties as compared to compositions of similar composition previously known and/or used in the art. Also provided herein are novel systems comprising these compositions and related compositions and methods of using such compositions and systems, such as in aesthetic treatment.

### CROSS-REFERENCE TO RELATED APPLICATIONS

This application claims the benefit of priority U.S. Provisional Patent Application 62/856,000, filed Jun. 1, 2019, the entirety of which is hereby incorporated by reference.

### FIELD OF THE INVENTION

This invention relates to novel compositions comprising resorbable polyester polymers, such as poly-L-lactic acid particles, including compositions that function as neocollagenic dermal injections and implants; systems comprising such compositions; and methods of using such compositions and systems, such as in aesthetic treatment.

### BACKGROUND OF THE INVENTION

A variety of products have been developed for human use in reparative or plastic surgery and in aesthetic dermatology; e.g., for the filling of wrinkles, fine lines, skin cracks, acne scars and other scars. Products that have been developed for such applications are often referred to as “dermal fillers.” A number of different materials have been used as dermal fillers in the past with varying degrees of success in their application.

Unfortunately, many dermal filler materials are not safe. For example, silicone gel (or silicone oil), is associated with a number of concerning properties including migration of droplets of silicone into tissues outside of the injection site; and because silicone is not biodegradable, it can be found at locations far from the site of injection, such as the liver. While silicone can be tolerated in many cases, side effects, including chronic inflammation, including formation of granulomas and allergic reactions, are often extreme and/or permanent. Because the side effects of silicone filler are known to be irreversible, it is banned for use in cosmetic applications in the European Union and the United States. Some amount of off-label use still reportedly occurs with claims of established ways of avoiding adverse effects. Teflon paste is another example of a dermal filler with concerning properties. This product, typically a suspension of polytetrafluoroethylene particles, ranging in diameter between about 10 and 100 micrometers (μm) in glycerine, has, in numerous cases, been associated with severe and chronic, serious infections and migration away from injection sites and has had to be removed after a few months from dermal and subdermal tissues for many patients. Bioplastics composed of polymerized silicone particles with a diameter of about 70 to 140 μm, typically dispersed in polyvinyl-pyrrolidone, have similarly been proposed, but are currently widely withdrawn as products/product candidates as clinical experience demonstrated that chronic inflammation and rejection reactions were commonly associated with use of such products. Alginate-derived fillers also have been associated with safety concerns (for example, the product Novabel® was pulled off of the market soon after launch due to granuloma formation).

Other products that have had better safety profiles have still proven to offer sub-optimal results. Collagen suspensions, for example, have been very widely used in the past; however, while safer than other products, results with collagen fillers have been disappointing since exogenous collagen is typically resorbed within one to four months. Such short-term efficacy presents a problematic user satisfaction and cost barrier. Allergic reactions also have been noted in about 2% of patients using collagen suspensions. Most collagen products are bovine derived, creating additional safety concerns. While believed to be safer and generally longer-lasting, human-derived or even autologous collagen products have not proven to be a strong alternative product to date. For example, autologous fat injections have been associated with high failure rates and significant expense. Calcium hydroxyl apatite, as used in the cosmetic filler Radiesse®, and acrylic hydrogel; polyacrylamide gel; polyalkylimide gel; silicone elastomer particles; and mineral oils, paraffin, and other lipid derivative-based products have also been used, but these products similarly have limited efficacy in aesthetic medicine applications and in some cases have also been associated with safety concerns.

Hyaluronate gels have proven to be an effective alternative to many of these other methods, by virtue of their biocompatibility and their lack of toxicity. Hyaluronic acid (HA) is present in all animal species and hyaluronate gels have been shown to be almost free of foreign proteins, hence lowering the risk for allergic reaction. Hyaluronic acid preparations are common; currently hundreds are commercially available. However, the rapid bioresorbability of HA products, which have an average maximum efficacy period of 2-6 months (depending on molecular size, cross-linking methods, and the injection location), has rendered many users dissatisfied with the performance of these products, especially in aesthetic applications.

More recently, there has been an interest in developing polymethyl methacrylate (PMMA) microspheres as dermal fillers. These products are composed of non-biodegradable microspheres contained in a suspension solution, typically of gelatin or collagen, and having a diameter ranging between about 20 μm to 40 μm. Insufficient time has passed in order to reliably understand the safety and efficacy of these products. Moreover, the delivery system for these products typically remains a solution of collagen of bovine origin, bringing known risks related to patient allergy as discussed previously. For example, PMMA beads suspended in bovine collagen marketed under the brand name Artecoll® (and more recently as Artefill® and Artesense®), have been reportedly associated with allergenic response concerns.

An alternative approach to the above-described dermal filler compositions has been to administer poly-L-lactic acid (PLLA) microparticle compositions. PLLA has been used for decades in medicine and surgery, but these products are a relatively new addition to aesthetic medicine. As opposed to true dermal fillers, these products lead to the generation of new, endogenous collagen (i.e., they are “neocollagenic”). Thus, the aesthetic effects of PLLA products have been demonstrated to last more than two years. SCULPTRA®, a product currently marketed by Galderma Laboratories for aesthetic applications (e.g., treatment of nasolabial folds, marionette lines and chin wrinkles), and approved by FDA more than 12 years ago, comprises PLLA microparticles as a primary functional component. SCULPTRA® is provided as two vials of lyophilized powder for reconstitution in water for injection before administration through a needle. Upon proper injection of rehydrated SCULPTRA®, collagen formation in the skin occurs resulting in long term filling effects. U.S. Pat. No. 6,716,251 discloses such PLLA injections for subcutaneous or intradermal injection. Although SCULPTRA® was developed by Aventis, one of the world's leading companies in healthcare product research and development, several problems and limitations associated with SCULPTRA® have limited its adoption and use, and little has been done to improve upon the product in the two decades since their initial introduction to the market, indicating that improvements in similar products are likely not expected without inventive new approaches and breakthroughs.

### SUMMARY OF THE INVENTION

The invention described herein provides novel compositions comprising particles of one or more types of resorbable polyester polymers (abbreviated as “RPP” elsewhere in this document), such as poly-L-lactic acid particles having new properties and characteristics (e.g., with respect to the size and shape of such particles and the consistency of such particles in terms of either or both of these factors). Typically, the compositions of the invention can be characterized as having a monodisperse collection of such particles. In several aspects, these particles can function as neocollagenic agents and, accordingly, form an active part of compositions that are suitable for use as dermal injections and/or implants. Also provided are novel systems comprising these compositions (such as needle and cannula delivery systems for injection of such compositions to mammalian recipients, such as human patients). Further provided are methods of making and using such compositions and systems, such as in the context of aesthetic modification/medicine.

In a specific exemplary aspect the invention provides dermatological injection/implant compositions comprising (a) a particle component that is composed of an effective amount of polyester polymer particles that are at least primarily composed of one or more resorbable polyester polymers (which typically have a monodisperse composition) and (b) a carrier component that is configured for (i.e., is suitable for) dermatological administration, wherein at least about 70% of the polyester polymer particles have a maximum particle diameter that is within (+/−) about 30% of the size of the mean particle diameter of the polyester polymer particles in the composition. In one aspect, about 65%, about 75%, or about 80% of the RPP particles have a maximum particle diameter that is within about 20% of the mean particle size or within about 35% of the mean particle size. In a preferred embodiment the particles also or alternatively are collagenic—i.e., they are capable of inducing a detectable amount of neocollagenesis following implantation into a suitable mammalian recipient, such as a human aesthetic treatment patient.

In aspects, the particles are typically primarily characterizable, if not essentially consisting of, microspheres. Because the microsphere compositions of the invention are typically monodisperse (at least about 87.5%, at least about 92.5%, at least about 95%, or at least about 97.5% of the particles have a maximum diameter that is within a range of less than about 20 μm, less than about 15 μm. less than about 12 μm, or less than about 10 μm) of each other, such RPP particles can also be referred to as “monospheres.” It should be understood that any aspect of this disclosure described in terms of particles or microspheres herein also provides support and disclosure, inherently, for monospheres.

Exemplary materials that can be used to make up RPP particles, such as monospheres, include polyglycolide (PGA), polylactic acid (PLA), polycaprolactone (PCL), and polyhydroxybutyrate (PHB) particles; copolymers of two or more thereof; and mixtures of any two or more thereof. Such compositions often include other excipients or components that can be considered a functional formulation, carrier, or vector, which can include surfactants, preservatives, and/or buffers. In some embodiments the composition includes gel forming agents. The formulation can be dried, such as through lyophilization (known alternatively as freeze-drying or cryodessication), and dried forms of such products can be reconstituted for ease of administration through needle and cannula systems. The compositions are often advantageously low in allergenicity and non-pyrogenic. In exemplary embodiments, the RPP particles and all functional, vector, or carrier elements of the formulation may be lyophilized with only water for injection used as the diluent for reconstitution. In alternative embodiments, only the RPP particles may be lyophilized, with all functional, vector, or carrier elements of the formulation being a component of the diluent in combination with water for injection for reconstitution.

In another aspect, the invention provides a neocollagenic dermatological product injection/implant delivery system comprising (a) a storage component containing an effective amount of a neocollagenic dermatological implant/injection composition having one or more of the above-described properties, particularly in terms of the shape, size, or both, and consistency thereof of the polyester polymer particles of the composition and (b) a needle or cannula configured to deliver the composition to the mammal upon application of a delivery force. The systems of the invention can be used with relatively less injection force than previously known, similar systems, and are associated with less injection failure than such prior art systems. The systems of such aspects also or alternatively are associated with relatively smaller needle sizes than those used to deliver current on-market products which lack the size, shape, and consistency of the polyester polymer particles of the invention (e.g., the system can comprise a 27G needle, a 28G needle, or a 29G needle). Storage components may be any suitable storage component capable of storing and delivering the implant/injection composition. The storage component may typically be a syringe or device comprising syringe components. The term syringe usually refers to a device or component comprising three main components/sub-components: (1) a barrel or storage component capable of holding the material for injection, for example a barrel; (2) a mechanism capable of pushing the material for injection out of the barrel upon application of a force to such mechanism, such as for example a plunger or piston; and (3) a connecting device being capable of attaching an implant/injection delivery device such as a needle or cannula to the material storage unit. Such a storage compartment may be a typical, manually operated syringe commonly utilized in the medical sciences. The term syringe also can be used to describe an alternative system capable of delivering an implant/injection composition via a needle, such systems having the components of a syringe, such as a material storage compartment, a means for expelling the material from the storage compartment, and a means for attaching the delivery device such as a needle to the storage compartment. Such systems may be characterized as, for example but not limited to, those having an electronic, motor, or computer controlled system; an device operated by pneumatic pressure; a device comprising a multi-syringe or multi-needle injection wherein multiple material housing components exist within the system; an ergonomically designed, manually operated device; a system with audible injected volume indicators; systems comprising a single-injection dose volume implant/injection composition storage component; systems capable of selectively setting an injection volume such that multiple injections of a precise volume may be administered (an adjustable injection syringe system); a pen-like device; or an adjustable injectable flow speed device; a fully automated and autonomous computer driven system. The invention also provides “kits” including the components of such delivery systems and compositions (e.g., a needle or cannula assembly, a lyophilized product comprising such particles and formulation or particles only, and a sufficient amount of water for injection or diluent containing water for injection along with other functional, vector, or carrier elements of the formulation for reconstitution).

In still other aspects the invention provides methods for delivering polyester polymer particles, which are preferably bioresorbable, safe, and neocollagenic, to a mammalian recipient, such as a human subject. In a particular exemplary aspect, the method is used in the performance of aesthetic augmentation or aesthetic treatment of a condition in a human subject. In an exemplary and preferred embodiment, the performance of such methods results in an aesthetic modification that is sustained over a substantial period of time (at least about 12 months, at least about 18 months, at least about 24 months, at least about 30 months, at least about 36 months, at least about 40 months, or longer), if not permanent, in the recipient, due to the stimulation of a detectable amount of new endogenous collagen in the patient in response to the injection or implantation of the particles.

In exemplary aspects, the invention may be used for aesthetic modification of the skin including but may not be limited to the aesthetic treatment of nasolabial folds (commonly referred to as “smile lines”) and areas of the chin and jawline including but not limited to marionette lines. The present invention may also be used to promote collagen genesis in the cheeks and temples, serving to address wrinkling, to soften bony areas, or to plump shallow areas of the face so as to promote a more youthful appearance.

These and several other aspects are described below. Any suitable aspect of this Summary of the Invention can be combined with any aspect described below.

### BRIEF DESCRIPTION OF THE FIGURES

FIG. 1 is a graph of injectability test results of SCULPTRA® samples using a 26G ⅝″ needle.

FIG. 2 is a graph of injectability test results of monodisperse PLLA microsphere test formulations using a 26G ⅝″ needle.

FIG. 3 is graphical overlay of the injectability measurements of SCULPTRA® versus the monodisperse PLLA microsphere test formulations using a 26G ⅝″ needle (i.e., an overlay of FIGS. 2 and 3).

FIG. 4 is a comparison of particle size between SCULPTRA® particles and monodisperse PLLA microspheres.

FIG. 5 is an accurate representation of a scanning electron micrograph image of SCULPTRA® particles.

FIG. 6 is an accurate representation of a scanning electron micrograph image of monodisperse PLLA microspheres of the invention.

### DETAILED DESCRIPTION OF THE INVENTION

The invention described herein provides, inter alia, neocollagenic dermatological injection/implant compositions that comprise (a) a particle component that is composed of an effective amount of polyester polymer particles, which particles are at least primarily composed of one or more resorbable polyester polymers, and (b) a carrier that is suitable for dermatological administration, wherein the particles of the composition are substantially uniform in size and shape (e.g., at least about 65%, such as at least about 70% or at least about 75% of the polyester polymer particles in the composition have a maximum particle diameter that is within (+/−) about 35%, such as about 30%, about 25%, about 25%, or about 20% of the size of the mean particle diameter of the polyester polymer particles in the composition).

We have determined that known microparticle products, including previously described polyester polymer microparticle products, e.g., SCULPTRA®, contain polydispersed particles of markedly inconsistent sizes and shapes, and often have a size coefficient of variation (CV) of more than about 20%. For example, in the experiments described herein, we have demonstrated that samples of SCULPTRA® have measured microsphere particle sizes in a single lot (#1A6112) with a CV of 42%. Large, irregularly shaped particles in SCULPTRA® (such size and shape disparity within SCULPTRA® shown in the scanning electron micrograph (SEM) provided herein), and similar compositions can lead to clogging of delivery systems, such as needles, and can be associated with other disadvantageous properties. We have discovered that by applying alternative methods of production to resorbable polyester polymer particles, it is possible to make compositions that overcome these shortcomings, while retaining and improving on the advantageous properties of such compositions, e.g., promoting neocollagenesis (the detectable stimulation of endogenous collagen production), in mammalian recipients such as humans undergoing aesthetic medical treatment, with such compositions.

Such particles, compositions comprising such particles, systems comprising such particles, and methods of making and using such particles, compositions, and systems are further described below, following a description of the principles of construction that the reader should consider in reading the disclosure provided herein.

### Principles of Construction

The following principles provide guidelines for understanding this disclosure.

All references, including publications, patent applications, and patents, cited herein, including the patents and patent applications cited above, are hereby incorporated by reference to the same extent as if each reference were individually and specifically indicated to be incorporated by reference and were set forth in its entirety herein. Accordingly, the reader should review and consider such references in understanding the full content of this disclosure. For example, unless clearly contradicted by context or explicit statement, the disclosure of such documents relating to formulations, methods of production, and methods of use of compositions and devices can be combined with the teachings provided herein to provide additional useful compositions and applications. However, the reader should understand that the citation and incorporation of patent documents herein is limited to the technical disclosure of such patent documents and does not reflect any view of the validity, patentability, or enforceability thereof.

All headings and sub-headings are used herein for convenience only and should not be construed as limiting the invention in any way. The use of the terms “a” and “an” and “the” and similar referents in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range within an order of magnitude of the order of the range, including the endpoints (e.g., a range of 1-2 is to be interpreted as providing support for 1.0, 1.1, 1.2, 1.3, . . . 1.9, and 2.0; a range of 10-20 is to be interpreted as providing support for 10, 11, 12, 13, . . . 19, and 20), unless otherwise indicated herein, and each separate value is incorporated into the specification as if it were individually recited herein. All ranges provided herein include the end points of the provided range, unless the exclusion of such endpoints is clearly stated or clearly indicated. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate—e.g., disclosure of “about 10” is to be understood as also providing support for 10 exactly). Terms of approximation, such as “about” are used herein where measurements are understood to vary due to measurement issues or variability in populations, such as results of clinical studies. The scope of such terms will depend on the context of the element at issue and the understanding of those skilled in the art. In the absence of such guidance in the art through relevant teachings or examples, “about” should be understood as meaning+/−10% of the indicated value(s).

As used herein, the singular form “a”, “an”, and “the” includes plural references unless clearly indicated otherwise.

Use of the term “or” herein is not meant to imply that alternatives are mutually exclusive unless clearly stated or clearly contradicted by context. Thus, in this application, the use of “or” means “and/or” unless expressly stated or understood by one skilled in the art and provides support for both such aspects.

All methods described herein can be performed in any suitable order unless otherwise indicated herein or otherwise clearly contradicted by context. Unless clearly indicated or contradicted by context, the elements of a composition disclosed herein (e.g., a pharmaceutical formulation) can be formulated in any suitable manner and by any suitable method. Unless otherwise explicitly stated or clearly contradicted by context, any combination of the various elements, steps, components, and/or features of the aspects of the invention described herein, and all possible variations thereof, is to be considered encompassed by the invention.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated. The breadth and scope of the present invention should not be limited by any of the above-described exemplary embodiments.

The description herein of any aspect or embodiment of the invention using terms such as “comprising”, “having,” “including,” or “containing” with reference to an element, composition, or set of compositions or elements should be interpreted, whether explicitly stated or not, as simultaneously providing support for a similar aspect or embodiment of the invention that “consists of”, “substantially comprises”, “predominately comprises”, “largely consists of”, and “substantially consists of” that particular element, unless otherwise stated or clearly contradicted by context (i.e., a composition described herein as comprising a particular element should be understood as also describing a composition consisting of that element, substantially comprising that element, predominately comprising that element, largely consisting of that element, and substantially consisting of that element, unless otherwise stated or clearly contradicted by context). Terms such as “including,” “containing,” and “having” should otherwise be interpreted openly herein, e.g., as meaning “including, but not limited to,” “including, without limitation,” or “comprising,” unless otherwise such a meaning is clearly contradicted. Any aspect described as including or comprising elements should be construed as “also or alternatively” including such elements along with the other elements of this disclosure.

“Substantially comprises” means that at least about 1% of a composition, population, or similar collection is or is made up of/by the referenced feature, species, or element, and typically means (and should be understood as providing support for) the relevant feature makes up or represents at least about 5%, at least about 10%, at least about 15%, and at least about 20% of the total amount of the composition or number of the population.

“Predominately comprises” means accounting for more than one half (i.e., more than 50%) of a feature (e.g., a composition or a population of things). This amount and similar amounts used in respect of defined terms provided herein can be on a weight percent (weight/weight) basis, on a molecule/molecule basis, or other relevant basis used in the context of the relevant disclosure. For example, if a composition is described as “predominately comprising” element/species A, more than 50% of the composition on a molecular and/or weight basis will be made up of element/species A). Wherever this term is used it should be understood as simultaneously providing support for more than 60%, more than 70%, and more than 80% of the component or composition or collection being made up of the feature, species, or element at issue. The term “most” should also be construed similarly herein.

“Largely consists of” means that at least about 75% of the composition, population, or the like is or is made up of the referenced feature, species, or element at hand and should be understood as providing disclosure that at least 82.5%, at least 87.5%, at least 92.5%, and at least 97.5% of the composition, population, or the like is or is made up of/by the referenced feature, species, or element. Obviously, the remaining minority portion of the relevant composition, collection, and the like can be composed of other compounds, materials, or other relevant elements. The phrases “largely all” and “largely most” should be construed similarly.

“Substantially consists of” means at least about 90% of the composition, population, or the like is made up of the referenced feature, species, or element and should be understood as also providing disclosure that at least about 95%, at least about 99%, or at least about 99.9% of the composition, collection, etc., is made up of the relevant element, feature, or thing. The phrases “nearly all” and “nearly entirely” should be construed similarly.

Changes to tense or presentation of phrases defined herein (e.g., using “comprises predominately” instead of “predominately comprises”) will not modify the meaning of the defined phrase, unless otherwise clearly indicated.

The use of section headings herein is for convenience in guiding the reader and breaking up the disclosure and is not to be interpreted as limiting the disclosure of the associated section or any other section.

The description of the specific embodiments provided herein will reveal the general nature of the invention such that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

### RPP Particle Composition

In one aspect, the invention described herein relates to novel compositions comprising (i.e., substantially comprising, predominately comprising, substantially consisting of, or consisting of) particles of resorbable polyester polymers (“RPPs”).

The RPP(s) that make up the particles of the compositions of the invention can be composed of known RPP materials, such as PLLA. The novel aspect of such compositions typically is found in, inter alia, the size of the RPP particles, the shape of the RPP particles, and the consistency of the particle composition in terms of one or both of these aspects.

The RPP particles of the invention can be used to prepare (and thus can be components of) a variety of compositions, which can, in turn, be used for a number of applications. Several applications of RPP compositions, such as PLLA microparticle compositions, are already known in the art, and the RPP particles provided herein generally can be used for any such uses (e.g., acting as delivery systems for pharmaceuticals, being used for 3D printer applications, acting as a substitute for PET (polyethene terephthalate) particles, and the like). For example, the RPP particle compositions of the invention can be used as dermatologic injections or implants, which is the application of primary focus herein. In one aspect, such dermatologic injections/implants can be characterized as being neocollagenic, i.e., as resulting in the detectable formation of new endogenous collagen following injection or other administration of the implant or injection (in some cases such new collagen formation is detected after two, three, four, or more months).

Particles provided by the invention and used in the compositions provided herein can also include other components in addition to one or more RPP components. For example, the particles can include one or more additional compositions that also are neocollagenic; lead to the stimulation of other endogenous biomolecules, such as elastin (i.e., are elastogenic), help to increase the health, volume, or other aspect of the skin of a recipient; or that otherwise assist in promoting the function of the particles.

In one respect, compositions of the invention can be characterized in comprising a particle component that substantially comprises, predominately comprises, or substantially consists of particles that consist essentially of one or more RPP materials that also or alternatively can be characterized in that such particles are (a) substantially uniform in size (e.g., are at least predominantly composed of particles that vary in size by no more than about 20%, such as no more than about 15%, or even no more than about 10% in maximum diameter or average maximum dimension, average dimension, and/or average minimum dimension), (b) substantially uniform in shape (e.g., wherein at least about 70%, at least about 80%, at least about 90% or more of the particles are spherical—having a diameter in every direction that varies by no more than about 10%, no more than about 5%, no more than about 2%, no more than about 1%, no more than about 0.5%, or no more than about 0.1% in distance), or (c) exhibit both substantial uniformity in terms of size and shape. The compositions can be deemed to “consist essentially” of the one or more RPP materials in that the particles provide or retain the desired function of the RPP particles, e.g., in terms of stable space filling, promoting endogenous collagen production, and/or one or more other desired structural and/or functional aspects associated with RPP particles. Thus, if such particles include additional materials—such as stabilizers, dermal fillers, or products that lead to the production of other endogenous biomolecules—such particles will still “consistent essentially” of the one or more RPPs contained therein because they have retained such fundamental features of RPP particles. This aspect of such compositions will be recognizable to one of ordinary skill in the art.

RPP particles are at least predominately composed of polyester compounds, and more commonly will substantially consist of, or entirely consist of (within levels of reasonable production and/or purification, if not also detection and/or isolation) polyester compounds.

The RPP particles are typically and usually preferably bioresorbable (or “resorbable”), meaning that they can, over a period of time, be broken down and assimilated into the body. In the case of RPP particles, resorption may occur over a number of weeks, more typically over a period of several months (e.g., about 2-6 months), but in some embodiments even years (e.g., about 1-3 years).

According to certain embodiments, the RPP is a homopolymer. According to some aspects the RPP is an aliphatic resorbable polyester homopolymer. It can be envisioned that other resorbable polymers comprising polyester monomer components mixed with other components, and having the other properties associated with particles of the invention (e.g., substantially uniform size, shape, and/or both) could be also or alternatively incorporated in the compositions and systems of this invention and/or used in the methods of the invention. RPP particles of the invention also or alternatively can be characterized as biodegradable, biocompatible, and/or biologically inert, as such terms would be understood in the art.

In one aspect, the invention provides compositions that comprise an “effective amount” of particles that comprise, substantially consist of, or consist of one or more types of RPP particles. The term “effective amount” means an amount that is effective for the intended application of the referenced composition, device, substance, or thing and/or that has been demonstrated to be effective for such an application in relevant testing (e.g., in one or more well-designed, conducted, well-controlled clinical studies, which are suitable for regulatory approval of a product). Thus, for example, an effective amount of a dermal implant/injection composition means an amount that is effective or that has been demonstrated to be effective in detectably modifying the condition and/or appearance of the skin or in causing a biological result, such as neocollagenesis. The phrase “effective amount” is used in such contexts to indicate that there is an amount of such particles present to lead to such modification or result, rather than there only being some insubstantial amount of such particles present or alternatively an amount that is too great to be suitable.

Numerous compositions described herein are characterized as “injection”, “implant”, or “injection/implant” compositions. In many aspects of the invention the compositions provided herein are suitable for administration to parts of the body of mammalian recipients, such as the skin of a human subject undergoing aesthetic modification. The particles of the invention can be delivered to such recipients/subjects by any suitable means. In most cases it is anticipated that delivery/administration of the composition will occur through either injection or implantation through other means. Particles can be injected in any suitable manner and numerous techniques for the injection of particles into the body, such as the skin, are known in the art. Typically, particles will be injected in an aqueous composition as a suspension through a suitable needle, examples of which are described elsewhere herein. Alternatively, the particles can be implanted by delivery through a cannula, surgical implantation, or other method of delivery. Except where features are described that are specific to one or the other, compositions for “implantation” or “injection” should be viewed as non-limiting and even providing support for each other, as in most cases the particles will be suitable for delivery by either technique.

The RPP particles typically are present in a composition. The composition can be any suitable composition for the storage, transport, and/or use (injection/implantation), of the particles contained therein. In one aspect, the other primary component of the composition is a carrier (which also may be described as a “carrier system” or “vector”). In most aspects, the carrier will be a carrier that is suitable for dermatological administration (e.g., implantation, injection through a needle, or both). Additional aspects of exemplary carrier systems are described elsewhere herein.

As further described elsewhere herein, the invention provides particle compositions wherein the particles “consist essentially” of one or more resorbable polyester polymers. That is to say, fundamental properties of such particles, such as causing neocollagenesis when delivered to the skin of a recipient leading to a sustained, if not permanent modification of the skin or its appearance, is maintained, even if the particles contain other features, compounds, or parts than the RPP(s) that make up some, most, or nearly all of the particles.

The RPP polymers often will be characterized as being neutrally charged, that is, having little positive or negative charge.

Most of the compositions of the invention are expected to be neocollagenic. To provide such a result, it is anticipated that typically the RPP particles of the invention will substantially comprise particles that are themselves neocollagenic (as determined by, e.g., testing substantially identical particles in clinical tests, in vitro assays, and the like, and producing the present particles to substantially similar or substantially identical specifications under substantially similar or substantially identical protocols).

RPP materials typically are synthetic (produced through chemical synthesis starting with either synthetic or natural materials, or both). Thus, such materials avoid inclusion of bovine and other animal-derived materials. A number of RPP products are, however, derived from plant materials (e.g., PLLA can be derived from plant material sources). In one context, RPP particles can be derived from such natural sources. In another context the RPP particles can be also or alternatively produced through chemical synthesis using synthetic starting materials.

As indicated elsewhere, a number of RPP compositions are known, and several have been used to varying degrees for aesthetic modification of humans. Such RPP materials can make up the most, if not all, of the RPP component of most if not all of the particles of any composition provided herein. In one aspect, the RPP material includes, substantially consists of, consists essentially of, or consists of (at least within the level of detection) a polyglycolide (a “PGA block”). Polyglycolides are known in the art and discussed elsewhere herein. In another aspect, the RPP material of the particles similarly is or includes a polycaprolactone (a “PCL block”). Such materials are also known in the art. In still another aspect, the RPP composition similarly is or includes a polyhydroxybutyrate (a “PHB block”), which is a material that is understood in the art. In still another aspect the RPP is or similarly includes a polylactic acid (or a “PLA”). PLAs are described and exemplified elsewhere herein and also are known in the art. The RPP particles also can include mixtures of two, three, or more of such materials and/or can include co-polymers formed from two or more of such materials. In some aspects, the RPP particles will substantially consist or consist of only one type of RPP material. In some aspects, RPP particles will substantially consist or consist of only RPP material. In some aspects, RPP particles will substantially consist or consist of RPP material that consists of only one type of RPP (e.g., the particles will consist entirely of a PLA, such as PLLA).

Suitable and sometimes advantageous examples of RPP materials include polylactic-co-glycolic acid (PLGA) materials, polylactic acid (PLA) materials, and materials that are composed of a mixture of such materials. In a particular aspect, at least 50% of the polyester polymer content of the composition is composed of (or consists essentially of) a polylactic acid (PLA) (e.g., at least about 65% of the particle content is composed of a PLA, at least about 80% of the content is composed of a PLA, at least about 90% of the content is composed of a PLA, or at least about 99% of the content is composed of a PLA).

A preferred RPP material in many contexts is a poly lactic acid (PLA). Poly lactic acid contains an asymmetric a-carbon which is typically described as the D or L form. It may also be described as R and S form. The enantiomeric forms of PLA are optically pure poly D-lactic acid (PDLA) and poly L-lactic (PLLA). PLA can be made in highly crystalline form (PLLA and PDLA) or it may be amorphous (poly D,L-lactic acid, PDLLA) due to atactically ordered polymer chains. In one aspect, the RPP particles comprise, substantially comprise, consist essentially of, substantially consist of, or consist entirely of PLA that is selected from poly-L-lactic acid (PLLA) (crystalline) or poly-D-lactic acid (PDLA) (crystalline), or a mixture thereof (PLLA and PDLA (homopolymer, crystalline) or PDLLA (copolymer, amorphous)). In one aspect, the RPP particles include or consist of such an amount of PLLA.

PLA polymers may range from amorphous glassy polymers to semi-crystalline and high crystalline polymers. It is known art to utilize different technologies to modify or enhance the mechanical properties of PLA. As non-limiting examples, annealing, adding nucleating agents, forming composites with fibers or nano-particles, chain extending, and introducing cross-linking structures are all ways of modifying the mechanical properties of PLA. Examples of such annealing technology may be found in Pramono et al, Polymer Degradation and Stability. 72 (2): 337-343 (2001); Horioshi et al. (2003), Polymer. 44 (19): 5635-5641; Tsuji, H. Polymer. 36 (14): 2709-2716 (1995); Hiroshi et al., Macromolecular Materials and Engineering. 288 (7): 562-568 doi: 10562-568 (2003); Trimaille et al (2003) Colloid and Polymer Science. 281(12): 1184-1190; and Hu et al., Macromolecular Materials and Engineering. 292 (5): 646-654 (2007). Examples of the formation of composites with fibers or nano-particles may be found in Hiroshi et al, Macromolecular Materials and Engineering. 288 (7): 562-568 (2003); Trimaille et al, Colloid and Polymer Science. 281(12): 1184-1190 (2003); and Hu et al, Macromolecular Materials and Engineering. 292 (5): 646-654 (2007). Examples of the chain extending technology may be found in Li et al. Polymers for Advanced Technologies. 17(6): 439-443 (2006); Di et al. Macromolecular Materials and Engineering. 290 (11): 1083-1090 (2005).

In an alternative embodiment, the RPP particles also or alternatively include, substantially consist of, consist essentially of, or simply consist of PLGA. PLGA is a copolymer of poly lactic acid (PLA) and poly glycolic acid (PGA). PLGA, is generally an acronym for poly D,L-lactic-co-glycolic acid, where D- and L-lactic forms are in equal ratio. PLGA is a biodegradable and biocompatible polymer capable of exhibiting a range of degradation times.

As is understood in the art and discussed elsewhere herein, the constituents of RPP co-polymers can be modified to change the properties of the RPP material. For example, PGA has no methyl side groups and has a higher crystalline structure than PLA; whereas the methyl side groups of PLA make it more hydrophobic than PGA. Therefore, PLGA copolymers richer in PLA are less hydrophilic, absorbing less water and as a result degrading more slowly in the body. “Tuning” of the, e.g., hydrophilicity, hydrolytic group interaction, crystallinity, and volume-to-surface ratio of the PLGA allows a user to dial in to degradation times ranging from less than one (1) month to more than six (6) months. See, e.g., Makadia and Siegel, Polymers (Basel). 2011 Sep. 1; 3(3): 1377-1397. doi:10.3390/polym3031377.

According to embodiments, a collection of RPP particles having the size and/or shape features of the various embodiments of the invention are provided wherein the RPP particles predominately comprise particles that are at least predominately composed of PLLA. In more particular aspects the invention provides compositions in which the particles largely consist of particles that at least largely consist of PLLA (e.g., at least 75% of the particles of the composition have a content that is at least 75% PLLA). In still other aspects, at least about 80%, at least about 90%, at least about 95% or more of the particles in the composition consist essentially of PLLA or substantially consist or consist of PLLA. PLLA RPP particles can be composed of any suitable type of PLLA and those of skill in the art can apply known principles to select and prepare PLLA materials having desired properties. See, e.g., Lasprilla et al., Biotechnology Advances 30 (2012) 321-328 (doi:10.1016).

According to embodiments, the RPP particles comprise, largely consist of, or substantially consist of and/or consist essentially of, or consist entirely of PLLA particles that are composed of poly-L-lactic acid (crystalline), a poly-D-lactic acid (amorphous), or a mixture thereof. PDLA typically is used in situations in which a shorter duration of the material is desired in the body.

The molecular mass of an RPP polymer, such as a PLA polymer, or more specifically a PLLA polymer, that makes up most if not substantially all of most, substantially all, or all of the particles of a composition of the invention, calculated by viscometry, is typically about 10,000-about 650,000 Daltons. According to more particular embodiments, the molecular mass of the polymer is about 50,000-about 250,000 Daltons. In still a more precise aspect, the polymer has a molecular mass of about 90,000-about 110,000 Daltons.

According to embodiments, the inherent viscosity of an RPP polymer, such as a PLA polymer, or more specifically a PLLA polymer, that makes up most if not substantially all of most, substantially all, or all of the particles of a composition of the invention is also or alternatively between about 0.1 dL/g and about 4.0 dL/g. In more specific embodiments the RPP polymer has an inherent viscosity of about 0.4-about 2.0 dL/g. In still a more precise aspect, the RPP polymer has an inherent viscosity of about 0.90-about 1.10 dL/g.

In a further aspect, the RPP polymer of the particles of the invention, as exemplified by PLA, may be a chiral polymer. In a more specific aspect, the polymer may be an optically active, levorotary polymer. In exemplary aspects, the polymer may be PLLA with a specific rotation of between about −150 and −160 cm²/g, commonly abbreviated to simply degrees (°).

In still another aspect, the RPP polymer of the particles of the invention, such as a PLLA used in most, substantially all, or all of the particles will have a melting point of between about 100° C. and about 300° C., such as between about 150° C. and about 200° C., such as 175-195° C., and in a more specific embodiment between 177 and 192° C. (e.g., 178.0-190.1° C.). Also or alternatively, wherein the heat of fusion (also referred to as enthalpy of fusion) of 100% crystalline PLLA is about 90 J/g, the RPP polymer of the particles in the present invention, such as PLA in most, having a mixture of crystalline (PLLA and/or PDLA) and amorphous (PDLLA) polymers, may have a heat of fusion ranging between about 25 J/g and about 150 J/g, such as between 50 J/g and 125 J/g, as in for example between about 60 J/g and about 95 J/g, for example more specifically between 65 J/g and 90 J/g.

Those of ordinary skill in the art will recognize that often particle compositions will not be completely pure due to normal aspects of the production process. Thus, for example, RPP particle compositions can contain some amount of residual solvents, residual monomers, or both. Residual solvents are leftover solvents from the manufacturing process, which are not removed, but that are considered to be present at an acceptable level. Residual monomers (e.g., lactic acid in the case of PLA) are monomers of a the RPP polymer that were not polymerized during the polymer production process, which usually also are present at a level considered to be suitable. The RPP polymer(s) used in the particles will according to some embodiments have be associated with less than 1%, less than 0.1%, less than 0.05%, or less than 0.01% residual solvents (e.g., less than 0.005% or less than 0.001% residual solvents). The RPP polymers used in the particles will also or alternatively be associated with less than about 2%, less than about 1%, less than about 0.5%, or less than about 0.1% residual monomer (e.g., lactic acid) (such as less than about 0.05%, less than about 0.025%, or less than about 0.01% residual monomer). Such percentages are also sometimes described as “ratios” herein.

Products that meet the various above-described features are known or can be produced using methods known in the art. For example, a PLLA product having the low residual monomer, low residual solvent, high melting temperature, and high heat fusion characteristics of aspects provided above is currently commercially available from Corbion (Gorinchem, The Netherlands) and Evonik Nutrition & Care GmbH (Essen, Germany).

The RPP polymers typically will biodegrade in vivo through nonspecific hydrolysis, typically releasing monomeric constituent(s) that are naturally endogenous and/or readily metabolized in a mammalian recipient, such as lactic acid, glycolic acid, or both compounds.

### Size Characteristics of RPP Particles

The RPP particles of invention typically can be characterized as being substantially uniform in size, shape or both. Thus, for example, RPP particles of the invention can be characterized in that, e.g., at least about 65%, such as at least about 70% or at least about 75% of the resorbable polyester polymer particles have a maximum particle diameter that is within (+/−) about 35%, such as +/−(or “within”) about 30%, within about 25%, or within about 20% of the size of the mean particle diameter of the polyester polymer particles present in the population of particles or the relevant composition in which such particles are contained.

RPP particles also or alternatively can be characterized as having a shape that is substantially uniform. The particles of this and other compositions of the invention can have any suitable shape. Shapes of particles may include disc shapes, diamond shapes, squircle shapes, and the like. However, in most cases the particles will be spherical or sphere-like (approximating a sphere or most closely resembling a sphere) or spheroid. Thus, for example, particles also typically can be characterized in that at least about 70%, at least about 80%, at least about 90%, at least about 95%, or at least about 99% of the particles have a diameter that varies by no more than about 35%, no more than about 30%, no more than about 25%, no more than about 20%, no more than about 15%, no more than about 10%, or no more than about 5% in any direction. According to embodiments, such particles may largely consist of, substantially consist of, or consist of microspheres, such that the particles will also or alternatively be characterizable as being substantially spherical. Thus, for example, the population of particles can be characterized in that at least about 65%, at least about 70%, or at least about 75% of the particles have, e.g., dimensions that are within about 2%, about 1%, about 0.5%, or even about 0.1% of all other dimensions. In one aspect, the RPP particles are substantially spherical, such that the RPP particles can be characterized as predominately comprising “microspherical” particles or “microspheres” (the term “micro” in this context indicating that the size of the particles being discussed is less than 1 mm, and typically less than 200 μm, 150 μm, or 100 um or the relevant composition or population of particles substantially consists of particles that are smaller than such indicated amounts). Such substantially similar or substantially identical diameter of such particles is an indicator that the particles are spherical (sometimes also informally described as “round” or “rounded”). However, in other aspects the particles can also or alternatively be determined to be spherical through other shape determining methods known in the art, including visual inspection under microscope, such as is demonstrated with respect to exemplary particles of the invention shown in the figures provided herein. Microspheres represent a preferred aspect of the invention. Accordingly, in every case where the term “particle” is used herein it is to be understood as also providing support for a similar aspect of the invention in which such particles are microspheres, unless such an understanding would be contradicted by disclosure, context, or suitability.

As already demonstrated, the substantially similar or substantially uniform size of the RPP particles is one important way to characterize the compositions of the invention. In one exemplary aspect the invention provides compositions in which at least about 75%, at least about 80%, at least about 85%, at least about 90%, at least about 95%, or at least about 99% of the polyester polymer particles of the composition have a maximum particle diameter that is within about 30%, within about 25%, or within about 20% (e.g., within about 5%, within about 2%, or within about 1%) of the mean particle diameter of the polyester polymer particles in the relevant collection/population or composition. Thus, an exemplary aspect of the invention is embodied in a neocollagenic dermal implant/injection comprising an effective amount of RPP particles, herein at least about 80% or at least about 85% of the polyester polymer particles of the composition have a maximum particle diameter that is within 20% (e.g., within about 25%) of the mean particle diameter of the polyester polymer particles in the composition. In another similar exemplary aspect, at least about 90% of the polyester polymer particles of the composition have a maximum particle diameter that is within 19%, within 18%, within 17%, within 16%, or within 15% of the mean particle diameter of the polyester polymer particles in the composition. In still another aspect, at least about 75% of the polyester polymer particles of a composition of the invention also or alternatively have a maximum particle diameter that is within 15% of the mean particle diameter of the polyester polymer particles in the composition. In another aspect, in at least about 85%, at least about 90%, at least 92.5%, or at least about 95% of the RPP particles the diameter in any direction varies by no more than about 25%, no more than about 20%, or no more than about 15%.

In some cases, the particles of the invention can be characterized by having measured size characteristics that fall within a desired measurement range. In one aspect, the particles of the invention can be characterized as having an average diameter and/or maximum diameter within the range of 10 μm to 200 μm, and more typically the particles of the invention will have a size that is characterized by average and/or maximum diameters that both fall within the range of about 20 μm to about 140 μm, about 20 μm to about 150 μm, or about 20 μm to about 100 μm. In still other aspects, the particles can be characterized in having a maximum diameter and/or average diameter within the range of about 25 μm to about 75 μm. Thus, for example, the invention provides compositions in which the particles of the composition largely consist or substantially consist of particles meeting these size features. In one aspect of the invention, most of the particles of the composition have a size defined by an average and/or maximum diameter that is less than about 60 μm, less than about 55 μm, or even less than 50 μm, such as for example less than about 40 μm, such as about 30 μm.

According to another aspect, the RPP particles of the composition also or alternatively can be characterized in including a limited/small number of particles (e.g., less than about 10%, less than 7.5%, less than about 5%, less than about 2%, less than about 1%, less than about 0.5%, or less than about 0.1%) that have a maximum diameter in any direction that is less than 20 μm in size. In one aspect the RPP particles of the composition may be characterized as comprising less than 5% of particles, such as less than 5%, less than 4%, less than 3%, less than 2%, or less than 1% of particles which are smaller than 10 μm in size, such as smaller than 9 μm in size, smaller than 8 μm in size, smaller than 7 μm in size, or for example smaller than 6 μm in size. In a further aspect, compositions are provided in which less than about 10%, less than 7.5%, less than about 5%, less than about 2%, less than about 1%, less than about 0.5%, or less than about 0.1% or the particles have a maximum diameter in any direction that is less than 5 μm such as less than 4 μm in size or for example less than 3 μm in size.

In aspects, at least about 75%, at least about 80%, at least about 85%, at least about 90%, at least about 95%, or at least about 97.5% of the particles are microspheres and at least about 40%, at least about 50%, at least about 60%, or at least about 70% of the microspheres have a maximum diameter within a range of 30+/−17.5 μm, 30+/−15 μm, 30 μm+/−12.5 μm, 30+/−10 μm, or 30+/−7.5 μm.

In a particular aspect, at least about 65%, at least about 70%, at least about 75%, at least about 80%, at least about 90%, at least about 95%, at least about 97.5%, or at least about 99% of the RPP particles in the composition have an average diameter and/or a maximum diameter that is about 10 μm to about 62.5 μm in size. In a more particular aspect, at least about 70%, at least about 80%, at least about 90%, at least about 97.5%, or at least about 99% of the RPP particles have a maximum and/or average diameter that is about 15 μm to about 60 μm in size. In a more particular aspect, at least about 60%, at least about 65%, at least about 70%, or at least about 80% of the RPP particles in the composition have a maximum particle diameter and/or mean particle diameter that is about 20 μm to about 57 μm in size. According to one aspect, such particles further predominately comprise PLA particles. According to one aspect, such particles further predominately comprise, largely consist of, substantially consist of, or consist entirely of PLLA particles.

In another aspect, a composition of the invention can also or alternatively be characterized by the fact that with respect to at least about 70%, at least about 80%, at least about 85%, at least about 90%, or at least about 95% of the polyester polymer particles in the composition the maximum diameter of any polyester polymer particle is no more than 300% the size of the minimum diameter of any other RPP particle detected (or of a minimum size detected in a set amount of particles such as at least 0.1%, at least 0.5%, or at least 1% of the particles). In still another aspect, the particles of a composition of the invention can also or alternatively be characterized by the fact that for at least about 40%, at least about 50%, at least about 60%, at least about 70%, or even for at least about 80% of the polyester polymer particles in the composition the maximum diameter of any polyester polymer particle is no more than 200% the size of the minimum diameter detected for RPP particles in the composition (e.g., the minimum diameter detected in an amount of particles estimated to make up at least 0.1%, at least 0.5%, or at least 1% of the particle in the composition).

In still another facet of the invention, the composition can be characterized in that the number of particles having a maximum diameter that is within about 30%, within about 25%, or within about 20% of the average maximum diameter of all RPP particles of the composition is at least two times (i.e., is at least 2× or 200%) as great (e.g., at least 2.25× as great, at least 2.5× as great, or at least 2.75× as great) as the number of particles that have a maximum diameter that differs from the average diameter by 150% or more, 200% or more, or 250% or more. In particular embodiments the number of particles within this uniform size distribution (within about 25%, within about 20%, or even within about 15% of the average maximum diameter of all particles) is at least 3 times as great as the number of polydisperse particles in the composition (e.g., the number of particles that have a maximum diameter that varies from the average particle diameter by 1.75×, 2×, 2.25×, or 2.5×). In a particular aspect, the number of particles of the composition that have a maximum diameter that is within 15% of the average maximum diameter is at least 2.5 times as great (e.g., at least 2.75×, at least 3.25×, or at least 3.5×) as the number of particles that have a maximum diameter that differs from the average maximum diameter by 250% or more.

Compositions of the invention can further be characterized in the maximum size of potential collections of agglomerated particles. Agglomeration of particles in a composition, such as a composition for injection, can be important with respect to use of such products in that it can, for example, lead to blockage of a needle or other delivery system, or lead to other undesirable effects. Because of the relatively small and relatively uniform or uniform size of the particles of the invention, the maximum size of agglomerates of the inventive RPP particles typically will be significantly smaller than agglomerates formed from particles of previously known products, such as SCULPTRA®.

According to an exemplary embodiment, the maximum average dimension of three agglomerated RPP particles of the inventive composition is less than 250 μm. In another aspect, the particles largely consist of or substantially consist of particles which, when three particles are agglomerated, have a maximum size of less than 200 μm (e.g., at least 95%, at least 97.5%, or at least 99% of the particles meet this limitation). In such cases the shape of the particles are such that even when agglomerated in a manner that aligns the largest dimension of all three particles the size of the formed agglomerate will be no more than 200 μm, 250 μm, or a similar measurement (e.g., no more than 180 μm, no more than 170 μm, or even no more than 160 μm or 150 μm).

In another facet, less than about 5%, less than about 3%, less than about 2%, or less than about 1% (e.g., less than about 0.5% or less than about 0.2%) of the RPP particles of the composition will have a maximum diameter that allows an agglomerate of three particles having the same maximum diameter to equal or exceed 250 μm in maximum diameter. In still another aspect, less than about 10%, less than about 5%, or less than about 2% of the RPP particles in the composition will have a maximum diameter that allows an agglomerate of three particles having the same maximum diameter to equal or exceed 200 μm or 180 μm in diameter. In yet another embodiment, less than about 5%, such as less than about 2.5%, or less than about 1% of the RPP particles of a composition also or alternatively will have a maximum diameter such that if the large diameter particle is agglomerated with two additional RPP particles having the average maximum diameter would result in an agglomerate with a maximum diameter that is equal or exceeding 250 μm, 200 μm, 180 μm, or 150 μm.

The compositions of the invention are expected to have measurably different characteristics from previously known compositions in terms of agglomeration. In low concentration suspensions, such as those described elsewhere herein, it is expected that the particles of the invention contained therein will exhibit markedly lower frequency and/or intensity/magnitude of agglomeration as compared to SCULPTRA® given the relatively smaller and significantly more uniform size of the particles, which in many embodiments will be spherical or substantially spherical. Thus, for example, the invention provides compositions in which the frequency of agglomerate formation in a solution with a comparable concentration of particles to SCULPTRA® is reduced by at least about 5%, at least about 10%, at least about 15%, at least about 20%, at least about 25%, at least about 35%, or more. For the same reasons, particles of the invention will be under different circumstances amenable to tighter and more efficient packing than the polydisperse particle composition of SCULPTRA®.

The RPP particles of the invention can be formed by any suitable means, techniques, or any suitable combinations of methods. Devices and methods for generating shaped particles, including microspheres, having uniform and relatively smaller size, and relatively uniform shape, and which can be used to produce RPP particles of the invention, such as RPP microparticles, are described in International Patent Application WO 2005/115599 (PCT/NL2005/000385).

In a particular facet of the invention the average RPP particle size of the composition is about 10 to about 60 μm is (typically reflected in the maximum diameter of such particles), such as about 20 to about 40 μm, e.g., about 25-about 35 μm.

According to embodiments, the RPP particle compositions or collections are typically monodisperse, having at least about 85%, at least about 90%, at least about 95%, or more (e.g., at least about 97.5% or at least about 99%) of the RPP particles having an average maximum diameter that is within about 15 μm, within about 12.5 μm, within about 10 μm, or within about 5 μm of one another. The compositions also or alternatively can be characterized by comprising at least 90%, such as at least 91%, at least 92%, at least 93%, at least 94%, at least 95%, at least 96%, at least 97%, at least 98%, at least 99%, or sometimes at least 99.5% of the particles (e.g., about 100%) of the composition have a particle size coefficient of variation (CV) less than about 50%, such as less than 45%, less than about 40%, less than 35%, less than about 30%, less than 25%, less than about 20%, less than 15%, less than about 12.5%, less than about 10%, or less than about 7.5%, such as less than 7%, less than 6%, or less than about 5%. In one aspect, the injection/implant composition may comprise particles having an average particle size ranging between 5 μm and 250 μm, and wherein the CV of the particle size within each of any such compositions is no more than 50%, such that it is less than 45%, less than 40%, less than 35%, about 30% or less, less than 25%, about 20% or less, about 15% or less, about 15% or less, or about 5% or less.

According to embodiments, the RPP material in the particles of the composition is also or alternatively predominately semi-crystalline or essentially all or all semi-crystalline. According to aspects, the composition is characterized by about 2-6%, about 3-5%, or about 4% crystallinity. According to facets, the molecular weight of the RPP material in the particles is about 70-200 kDa, such as about 80-160 kDa, or about 90-120 kDa, such as about 110 kDa. The RPP particles also or alternatively can be characterized in that they have less than about 3%, less than about 2%, or less than about 1% water content (e.g., less than about 0.5% water content). The compositions can be further characterized in having a DCM residual level of less than about 700 ppm, less than about 650 ppm, less than about 600 ppm, less than about 550 ppm, or less than about 500 ppm.

According to certain facets, the mean particle size of particles of compositions having one or more of the above-described features also or alternatively does not decrease by more than about 10%, such as by more than about 8%, by more than about 6%, by more than about 4%, by more than about 2%, or in some aspects by more than about 1%, or in some aspects by even more than about 0.5% over the course of a significant time period (e.g., at least 3 months, at least 4 months, at least 6 months, or longer), under typical long term/normal or accelerated stability conditions used in pharmaceutical/device testing. In aspects, particles of a composition do not exhibit a decrease in size of more than about 10% (e.g., more than about 8%, 6%, 4%, 2%, or 1%) when maintained (a) at about 40° C. (e.g., 38-42° C.) at about 75% (e.g., 70-80%) relative humidity (RH) for 6 months (representative of a typical device/drug accelerated stability study); (b) when maintained under typical long term/normal stability conditions (e.g., at about 25° C. (e.g., ±2° C.) and about 60% RH (e.g., ±5%), at about 30° C. (e.g., ±2° C.) and about 65% RH (e.g., ±5%), or both, for 12 months or longer (e.g., 18 months or 24 months), or (c) under both long term/normal and accelerated stability testing conditions. Any disclosure herein concerning accelerated stability testing conditions implicitly provides support for also or alternatively achieving the same results under normal/long term stability testing conditions.

### Stability

In some aspects, the RPP particle formulations described herein are capable of being stably sustained for significant periods of time, e.g., at least about 3 months, 4 months, 6 months, 12 months, 18 months, 24 months, or longer, under (a) typical storage or stability testing conditions, (b) accelerated stability testing conditions (which are considered indicative of long term storage capability), or (c) both of such conditions (specific aspects of which are set forth above and are known in the art). In an exemplary aspect, particles maintained under accelerated stability conditions of, e.g., about ° C. and 75% relative humidity, can be maintained for 3, 4, or 6 months without loss of suitability for use or significant change in the size characteristics of the particles. In certain facets, the average molecular weight of the particles (Mw, in kDa) maintained under accelerated storage conditions of e.g., 3-12 months (e.g., 4-8 months) or real time storage of 12-36 (e.g., 18-30) months, or both, varies by less than about 5% from an initial time point (t=0) to six months when held at such conditions. In aspects, the average molecular weight of particles held under such conditions for such periods varies by less than about 4.5%, less than about 4%, less than about 3.5%, less than about 3%, less than about 2.5%, less than about 2%, less than about 1.7%, less than about 1.5%, less than about 1.3%, or less than about 1%, such as in certain aspects less than about 0.5% or less than even about 0.1%.

In certain aspects, the RPP particle formulations can maintain an average molecular weight of between about 105 kDa and 115 kDa, such as between about 106 kDa and about 114 kDa, for at least 3, 4, or 6 months when maintained under accelerated stability conditions (e.g., 40° C. and 75% relative humidity), when held under prolonged normal storage conditions (of room temperature and typical humidity for 12-36 months, e.g., 18-30 months), or both.

In some aspects, the polydispersity index (PDI) (a measure of the breadth of the molecular weight distribution), in particle compositions varies over the course of storage/stability testing, over similar periods and conditions as those described above (e.g., for 6 months at 40° C. and 75% relative humidity), by less than about 5%, such as for example by less than about 4.5%, less than about 4%, less than about 3.5%, less than about 3%, less than about 2.5%, or less than about 2%, such as less than about 1.5%, or in some cases even less than about 1%. In some facets, the RPP formulations described herein can maintain an average PDI of between about 1.30 and 1.40, such as between about 1.20 and about 1.38 for at least six months when held at 40° C. and 75% relative humidity.

According to some facets, RPP particle formulations described herein are capable of maintaining a degree of crystallinity when held under accelerated storage conditions or long term ordinary storage conditions, as exemplified above (e.g., at 40° C. and 75% relative humidity for 6 months), which varies by no more than approximately 20%, such as for example by no more than about 18%, no more than about 16%, no more than about 14%, or for example by no more than about 12% or no more than about 10%. In certain aspects, the RPP particle formulations described herein can maintain an average degree of crystallinity of between about 3 and about 12, such as between about 4 and about 11, for at least six months when held at 40° C. and 75% relative humidity.

In certain aspects, the RPP particle formulations described herein are capable of maintaining a d10 (that is, a population of particles wherein 10% of the particles have a smaller diameter than a given value) when held under accelerated stability conditions (at 40° C. and 75% relative humidity for 6 months), ordinary/long term storage conditions, or both, which varies by less than 5%, less than about 4.5%, less than about 4%, less than about 3.5%, less than about 3%, less than about 2.5%, less than about 2%, less than about 1.5%, less than about 1%, or in certain aspects, less than even about 0.5%.

In some facets, the RPP particle formulations described herein are capable of maintaining a d50 (that is, a population of particles wherein 50% of the particles have a smaller diameter than a given value) when held for at 40° C. and 75% relative humidity for 6 months (or under normal/long term storage conditions for at least 12 months, or both) which varies by less than 5%, such as less than about 4.5%, less than about 4%, less than about 3.5%, less than about 3%, less than about 2.5%, less than about 2%, less than about 1.5%, less than about 1%, or in certain aspects, less than even about 0.5%.

In some aspects, the RPP particle formulations described herein are capable of maintaining a d90 (that is, a population of particles wherein 90% of the particles have a smaller diameter than a given value) when held for at 40° C. and 75% relative humidity for 6 months (or under normal/long term storage conditions for at least 12 months, or both) which varies by less than 60%, such as for example less than about 50%, less than about 40%, less than about 35%, less than about 30%, less than about 25%, or less than about 20%, such as for example less than about 15%, less than about 10%, less than about 5%, less than about 4%, less than about 3.5%, less than about 3%, less than about 2.5%, less than about 2%, less than about 1.5%, or in certain aspects less than about 1%.

In certain facets, the RPP particle formulations described herein are capable of maintaining a mean diameter when maintained under such long term regular or accelerated stability testing conditions (e.g., when held at 40° C. and 75% relative humidity for 6 months), ordinary/long term storage conditions, or both, which varies by less than about 50%, such as for example by less than about 45%, less than about 40%, less than about 35%, less than about 30%, less than about 25%, or less than about 20%, such as for example less than about 15%, less than about 10%, less than about 5%, less than about 4.5%, less than about 4%, less than about 3.5%, less than about 3%, less than about 2.5%, less than about 2%, less than about 1.5%, or in certain aspects less than about 1% or less than about 0.5%.

According to certain facets, the RPP particle formulations described herein can maintain a d10 value of between 25 μm and 30 μm when held under such long term ordinary or accelerated stability testing conditions (e.g., when held for at least six months when held at 40° C. and 75% relative humidity), ordinary/long term storage conditions, or both. In some aspects, the RPP particle formulations described herein can maintain a d50 value of between 25 μm and 35 μm for at least six months when held at 40° C. and 75% relative humidity (or under other stability testing conditions—e.g., 2 years at RT). In some aspects, the RPP formulations described herein can maintain a d90 value of between 70 μm and 75 μm for at least six months when held at 40° C. and 75% relative humidity. In some aspects, the RPP particle formulations described herein can maintain a d90 value of between 30 μm and 35 μm for at least six months when held at 40° C. and 75% relative humidity. In some aspects, the RPP particle formulations described herein can maintain an average mean diameter of between 38 μm and 40 μm for at least six months when held at 40° C. and 75% relative humidity. In some aspects, the RPP formulations described herein can maintain an average mean diameter of between 28 μm and 32 μm for at least six months at 40° C. and 75% relative humidity.

### Carriers and Compositions

As noted elsewhere herein, RPP particle compositions of the invention can be combined with other compounds to form compositions having desirable properties. For example, particle compositions of the invention can be combined with a carrier system (“carrier” or “vector”) to generate a suspension (or “formulation”) comprising the particles, a suitable base carrier or diluent (typically water, such as water for injection (WFI)), and, optionally, one or more additional functional/structural compounds that enhance the performance of the suspension/formulation (“excipients” or “additives”).

Examples of excipients and additives for injectable and implantable products, such as dermatologic fillers, are well known in the art, and, in general, any safe and suitable excipient or set of excipients can be combined with the RPP particles of the invention to enhance the performance of the resulting composition in the context of its intended use.

In one exemplary embodiment, the invention provides compositions comprising an effective amount of RPP particles according to any of the various aspects described herein in combination with (a) an effective amount of one or more physiological acceptable surfactants and (b) an effective amount of one or more physiologically acceptable viscosity-increasing and/or gelling agents, such that the combination of the excipients and particles can form a viscous solution and/or a gel. It will be understood that any formulation described herein can be a viscous solution comprising a dispersion of particles, a gel, or any other suitable form of formulation, which typically will be a liquid, which may be, e.g., a gel. Furthermore, agents which increase the viscosity and/or gelling agents may be incorporated in the compositions or methods of the invention. In some cases, viscosity-enhancing agents may also be referred to as “gelling agents” in the art or understood to be within the category of compounds referred to as “gelling agents” herein.

The excipient components of the formulation typically is resorbable within about six months or less, within about four months or less, within about three months or less, within 2.5 months or less, or within two months or less (e.g., within six-12, such as seven-10 weeks) (e.g., as may be determined to be the case in at least about 95% of such liquids or gels, at least about 99% of such gels, or at least about 99.9% of such liquids or gels when tested under repeatedly conditions, such as are used in medical device/pharmaceutical quality control procedures known in the art and/or through clinical testing in one or more well-controlled, well powered, and otherwise adequate studies). The particles, however, typically will be retained in the body for longer periods, e.g., at least about six months, typically at least about 12 months, at least about 18 months, or even about two years (e.g., on average as determined by administration to a population of subjects in a well-controlled, adequate clinical trial).

Liquid formulation compositions according to typical aspects are mostly composed of water for injection (sterile water typically produced through reverse osmosis and having other characteristics known in the art). Such compositions can be considered dispersions or suspensions of the particles in a liquid, which sometimes is a viscous liquid, and in other cases is a gel. In general, disclosure of any type of formulation can be interchanged herein. The terms liquid and gel are sometimes used separately to highlight that formulation compositions of the invention can be non-gel liquid formulations, such as a dispersion of the particles in an aqueous liquid composition. In specific aspects, the formulation composition will also compose one or more gelling agents, which typically are authorized in injection formulations by relevant regulatory agencies. In one facet, the invention provides RPP particle gels or liquids that comprise an effective amount of one or more cellulose derivatives, such as a carboxymethylcellulose (CMC). The amount of such material can be an amount that is effective for providing the desired property in the composition (i.e., an “effective amount”). A range of amounts may, for example, result in formation of a gel, whereas a narrower range will provide a gel with a certain viscosity and/or other rheology characteristics, but in other aspects the formulation will remain a non-gel liquid. In one exemplary aspect, the invention provides a liquid or gel formulation comprising a CMC at a concentration by mass of about 0.1 to about 7.5%, such as from about 0.1 to about 5.0%. In another aspect, the gel also or alternative comprises hydroxypropyl-methylcellulose (HPMC). Other formulation components can also or alternatively include, e.g., hyaluronic acid, chitosan, collagen, other gelling agents, e.g., gelling agents capable of forming, supporting, and/or maintaining a solid colloidal suspension. According to embodiments, compositions of the invention can also or alternatively also comprise hyaluronic acid (typically synthetic HA) and/or esters, such as lactic acid esters or caproic acid esters.

In another embodiment, dispersion of the RPP particles/microspheres of invention and/or the homogeneity of the liquid or gel will be provided or enhanced by the use of an effective amount of one or more surfactants. Surfactants used in such compositions will typically be selected based on innocuousness, and usually will be agents that are authorized subcutaneous and intradermal use by regulatory authorities, such as the US FDA. The surfactant will typically also or alternatively be a nonionic surfactant. Polyoxyethylene sorbitan monooleate compositions are examples of such suitable surfactants. Such surfactants are known in the art and, e.g., marketed under the name Tween®, such as Tween® 80. Other known surfactants include the Pluronic/poloxamer surfactants (see., e.g., H. H. Bearat and B. L. Vernon, in Injectable Biomaterials, 2011). Other possible surfactants include but may not be limited to those with similar characteristics to Tween 80, such as other nonionic surfactants with a similar viscosity (about 300-500 centistokes at 25° C.), similar molar mass (about 1310 g/mol), similar density (1.06-1.10)), and/or similar pH (pH of a 5% aqueous solution being between 6 and 8), which can impart a comparable ability to provide sufficient dispersion of the microspheres and to maintain the homogeneity of the liquid or gel. In still another aspect, compositions also or alternatively comprising polysorbate 20 (e.g., Tween® 20) or its equivalent are provided.

In certain embodiments, the surfactant may be selected from any number of surfactants which are useful in providing for homogeneity of dispersed particles/microspheres in a formulation composition. Such surfactants may be non-ionic surfactants other than Tween 80, such as other sorbitan surfactants, e.g. SPAN™ 80, SPAN™ 85, SPAN™ 65, SPAN™ 60, SPAN™ 40, SPAN™ 20, TWEEN® 40, TWEEN® 20, TWEEN® 21, TWEEN® 60, Triton-X® 100, or any mixture thereof. In other embodiments, the non-ionic surfactant may be an alkyl glucoside, or PEG-400.

In yet further embodiments, the surfactant component of a formulation may be a non-ionic surfactant having a molecular weight of from about 100 Daltons to about 2,000 Daltons. In one or more embodiments, a non-ionic surfactant of the present invention may have a molecular weight from a low of about 100, about 200, or about 300 Daltons and/or a high-end molecular weight of about 2500, about 2000, about 1500, about 1300, about 1200, or about 1,000 Daltons.

Another aspect the invention provides a composition of RPP particles, wherein the composition is in the form of an aqueous liquid or a gel. In such aspects, a formulation composition typically comprises one or more physiologically acceptable buffers, one or more physiologically acceptable salts, one or more physiologically acceptable preservatives, or a combination of two or more thereof. In one set of exemplary aspects, compositions are provided that comprise at least about 90% water, about 0.05-2% of a preservative, such as citric acid, about 0.05-0.75%, such as about 0.05-0.25% of salts and/or buffers (e.g., disodium hydrogen phosphate dihydrate, sodium chloride, and/or sodium hydroxide), and about 1-5%, such as about 1.5-about 4.5%, such as about 1.75%-about 3.5%, e.g., about 2-3% of a gelling agent, such as CMC or HPMC. Physiologically acceptable buffers, physiologically acceptable salts, and physiologically acceptable preservatives are types of excipients known in the art. Exemplary buffer components, salts and preservatives which may be used in any formulation composition are exemplified elsewhere herein.

Excipients and particles of any formulation composition are typically selected and configured such that the effect of implantation/injection of the particles is detectable in human recipients for at least about 6 months, at least about 12 months, at least about 18 months, at least about 24 months, or longer (e.g., at least about 27 months, at least about 30 months, or at least about 36 months) in human recipients (e.g., on average as determined in clinical testing in one or more well controlled, adequate studies).

A formulation composition of the invention will desirably be safe for administration to a human recipient. In one facet, the invention provides formulation compositions in which a detectable allergenic reaction occurs in less than about 1%, such as less than about 0.5%, or less than about 0.1% of human recipients (e.g., as determined through clinical study). In another aspect, the invention provides formulation compositions that also or alternatively can be classified as a “non-pyrogenic”, resulting in a detectable inflammatory reference in less than about 1%, such as less than about 0.5%, or less than about 0.1% of human recipients (e.g., as determined through one or more clinical studies).

Formulation compositions of the invention and other compositions of RPP particles provided herein can be of any suitable volume and can comprise any amount of RPP particles that will be effective for the intended application of the composition.

In one aspect, the invention provides compositions that comprise 100 mg to about 300 mg of one or more RPP particles. In another facet, the invention provides compositions that comprise 125 mg to about 175 mg of RPP particles. In still a more particular aspect, the invention provides compositions that contain about 140 mg to about 160 mg of RPP particles. In an even more specific facet, the invention provides compositions that comprise about 145 mg to about 155 mg of one or more RPP particles, such as PLLA particles. Such compositions may or may not include other excipients.

In aspects, compositions comprising excipients and RPP particles, but without water or other diluents, are provided. Such dried compositions can be useful for, e.g., transport and storage of particle compositions. In such cases, the excipient/particle formulation can be reconstituted with a diluent, such as water for injection (WFI), prior to injection or implantation. In some aspects, a sugar alcohol, such as mannitol, can be used as a diluent and/or as a lyophilization aid. For example, a reconstituted/wet composition of the invention can comprise about 0.5%-about 2.5% of mannitol (w/w), in some aspects. Such compositions can and will typically comprise about 200 mg to about 500 mg of a dried formulation comprising excipients and RPP particles, such as about 225-450 mg of a dried formulation, e.g., about 250-425 mg of dried formulation, such as about 275 mg to about 400 mg of dried formulation, or about 300-about 400 mg or about 350-about 400 mg of a dried formulation of excipients and particles.

In aqueous suspension/formulation compositions of the invention, the particles can be present in any suitable concentration. In one aspect, the invention provides suspensions in which the concentration of RPP particles in the suspension is about 10-about 30 mg/mL. In a more specific aspect, the concentration of RPP particles in such a suspension is about 15-25 mg/mL. In an exemplary facet, the concentration of RPP particles in a composition is about 20 mg/mL.

The concentration of particles can also be characterized on a weight percent basis, which can be especially useful when considering the concentration of the RPP particles in dried formulations. In one exemplary aspect, the concentration of polyester polymer particles in a composition (without water or diluent) is about 25% to about 50% (w/w). In a more particular aspect, the concentration of the RPP particles in such a dry formulation composition is about 30%-about 42.5%, such as about 35%-about 40%.

Dried particle formulations can be generated by any suitable means. In one aspect, the dried formulation is prepared by freeze-drying (lyophilization) applied to a formulation comprising the excipients and the particles.

Reconstituted formulations can include any suitable amount of WFI or other diluent. In one aspect, compositions comprising about 3.5-about 10.5 mL water for injection or other suitable diluents are provided. In a more particular aspect, such a composition comprises about 4-about 9 mL water for injection or another suitable diluent. In still an even more particular aspect, the composition comprises about 4.5 mL to about 8.5 mL water for injection or suitable replacement diluent.

The formulation in the reconstituted composition can have any suitable concentration. In one aspect, the concentration of the formulation (comprising both the particles and the excipients) in the liquid or gel formulation composition is about 45-about 65 mg/mL. In more particular facets, this concentration is about 50-about 60 mg/mL, such as about 51-about 55 mg/mL. In one facet, the concentration of the formulation (non-water constituents, that is, RPP particles plus excipients) in the aqueous gel or liquid composition (final, reconstituted formulation) is about 3.5 wt. %-about 7.0 wt. %, such as about 4.5 wt. %-about 6.0 wt. %, for example about 5.0 wt. % to about 5.5 wt. %. The concentration of RPP particles in a resuspended/dilute formulation will typically be about 0.5 wt. %-about 5 wt. %, such as about 1 wt. %-about 4 wt. %, such as about 1.5 wt. %-about 2.5 wt. % (e.g., about 1.8 wt. %, about 2 wt. %, or about 2.2 wt. %).

Compositions of the invention also can be characterized on the basis of the concentration of particles in the composition. Typically, the concentration of RPP particles in a fully re-hydrated, gel or liquid composition will be in the range of about 1.5 wt. % to about 2.5 wt. %, e.g., about 2%. In more particular aspects, the concentration of RPP particles is about 1.9 wt. % to about 2.1 wt. %. The concentration of the RPP particles in the formulation (when dried/undiluted; that is, the percent RPP of all non-water constituents) may, however, range in some aspects from about 20% to about 60%, such as about 35%-about 70%, about 35%-about 50%, or about 40%. The total amount of RPP particles present may be, e.g., about 100 mg to about 300 mg, such as about 125 mg to about 250 mg, such as about 150 mg.

Reconstituted or otherwise liquid formulation compositions of the invention intended for injection of particles are desirably “syringeable.” “Syringeability” refers to ease of administration via syringe of the product, as may be determined by user acceptance studies and the like. In one aspect, the syringeability of a composition of the invention is determined by user study or user feedback to be at least about 10%, at least about 15%, at least about 20%, at least about 25%, at least about 30%, or even at least about 35% greater than the level for SCULPTRA®.

According to certain embodiments the amount of formulation in the composition (excipients and RPP particles) will range from about 100 mg to about 600 mg, such as about 250 mg to about 500 mg, such as about 350 mg to about 450 mg. According to aspects, the formulation of the particles and excipient in a composition will be about 30-about 90 mg/mL, such as about 35-85 mg/mL

The gelling agent when present will typically make up about 30% to about 90% of the excipients, such as about 35% to about 80% or about 40% to about 75%. This concentration of gelling agent in a combined dried formulation (with RPP particles) will typically be reduced to about 15%-about 75%, such as about 35%-about 55% (w/w), for example about 40 to about 50% (w/w). A weight/volume % of such a gelling agent typically will be between about 1% and about 3% in the final wet formulation. The weight/weight percent of the gelling agent in a reconstituted formulation will be in the same range (about 1-about 2.5%). In a typical wet formulation, about 75 to about 275 mg of gelling agent (e.g., CMC) is present, such as about 100 to about 250 mg of gelling agent, for example about 150 to about 200 mg of gelling agent, for example about 170 to about 180 mg.

The surfactant, if present, will typically make up about 1.0-about 5% (w/w) of the excipient composition, for example about 2.0 to about 4.0% (w/w), such as about 2.5% to about 3.5% of the excipient composition (w/w). The surfactant, if present, will typically make up about 0.5-about 3.5% of the final dried formulation (excipients plus RPP particles) e.g., about 1.5 to about 2.5% or about 1.75 to about 2.0% (e.g., about 1.9%) of the final dried formulation (RPP particles plus excipients) (w/w) and about 0.05% to about 0.2%, such as about 0.1% of the concentration (w/v % and/or w/w %) of the final, wet formulation. A composition of the invention can comprise about 5 mg-about 10 mg of a nonionic surfactant, such as a polysorbate or pluronic surfactant. In more particular embodiments, the composition will comprise about 6-about 9 mg, such as about 7.5 mg of a surfactant.

Particles in compositions of the invention will typically exhibit a resorbability time of between about 1 and about 3 years when injected into human tissue, such as skin. In more particular aspects, the resorbability time of the RPP particles in the formulation is between about 1.5 and about 2.5 years.

### Delivery Systems and Kits

In addition to the compositions described above, the invention also provides delivery systems and kits that comprise one or more of such compositions.

In one exemplary embodiment, the invention provides an implant/injection delivery system that comprises a device including a storage component, which typically will contain an effective amount of a neocollagenic RPP particle composition described above, and a needle, cannula, or other delivery component through which the composition can be delivered into the body of mammalian recipients, such as into the skin of a human aesthetic medicine patient. In the case of an injection device, the device is configured to deliver the composition upon application of a delivery force.

Typically, the amount of RPP particles contained in the delivery system will be in an amount of about 100-about 600 mg, such as about 125 mg to about 500 mg, e.g., about 135 mg to about 400 mg. In a particular aspect, the amount of RPP particle material is about 100 mg-about 200 mg, such as about 125 mg-about 175 mg, e.g., about 150 mg. Ordinarily, such RPP particles are contained in an aqueous gel or liquid, which contains about 3.5 mL-about 10.5 mL, e.g., about 4-about 9 mL of diluent (e.g., WFI) and excipients.

Needle delivery devices are an important aspect of the invention. In one aspect the invention provides a needle delivery system wherein the inner diameter of the needle is about 0.1 mm to about 0.4 mm in size. In another aspect, the inner diameter of the needle delivery system is about 0.125 mm to about 0.285 mm. In yet an even more particular aspect, the inner diameter of the needle is about 0.11 mm to about 0.35 mm. The invention also provides needle delivery systems wherein the outer diameter of the needle is also or alternatively about 0.1 mm-about 0.7 mm, such as about 0.2 mm to about 0.6 mm, or more specifically between about 0.23 mm to about 0.59 mm. According to certain aspects, a needle delivery system capable of successfully delivering the RPP particle compositions described herein is one comprising a smaller inner diameter, such as for example an inner diameter that is at least about 1% smaller, at least about 3% smaller, at least about 5% smaller, at least about 10% smaller, or even more, such as at least about 12% smaller, about 14% smaller, about 16% smaller, about 18% smaller, or even about 20% smaller or more than the inner diameter of a needle delivery system required to deliver an on market product for a similar purpose, such as for example SCULPTRA®. In certain aspects, the ability to use a needle delivery system having a smaller inner diameter allows for a corresponding reduction in outer diameter needle dimensions, thus in some aspects providing an improved patient experience due to the ability to utilize reduced needle diameter dimensions to pierce the skin during administration compared to on market products such as SCULPTRA®. In aspects, the maximum force, minimum force, or both the maximum and minimum force required by a typical user (e.g., as determined through product use trials) used deliver an amount/dosage of a particle composition through a needle (e.g., a 26G or 28G needle) is less than about 75%, e.g., less than about 66.6%, such as less than about 50%, e.g., less than about 33%, or less than about 25% of the force required to deliver a corresponding amount of SCULPTRA® through the same sized needle.

A number of needles that are suitable for injection of such compositions are known in the art. Needle outer diameter is typically measured by gauge, and inner diameters typically increase/decrease correspondingly in size, as is understood in the art. In one aspect of the invention, the delivery system includes all needles and/or cannulas ranging between 24G and 31G. In more particular aspects, the needle system comprises a 26G needle, a 27G needle, or a 28G needle. In one aspect, the needle is a needle that is smaller than an average 26G needle in inner and/or outer diameter, such as being one of the smaller diameter needles listed here or by being otherwise at least about 10%, at least about 15%, or at least about 20% smaller in inner and/or outer diameter.

A delivery system can comprise a cannula as an alternative to a needle delivery system. Cannula systems suitable for delivery of materials such as dermal fillers are known in the art. Thus, for example, the formulation compositions described herein may be injected with a blunt-tip dermal filler microcannula. Such microcannulas present an alternative to needles for the injection of dermal fill products and may be preferable in certain circumstances. As an example, microcannulas may provide access to a large treatment area from a single injection point, as the cannula may be moved around beneath the skin with less risk of damage to vasculature. In addition, needles require multiple injection points. As an example, an injection in the cheek using a microcannula may reach treatment areas from the cheek to the outer edge of the face; from two points on corners of the mouth, a dermatologist may be able to treat the upper lip, lower lip, and nasolabial folds as well as marionettes lines. Using a needle for injection, additional injection points would be required. Other circumstances where injection via a microcannula versus a needle may be advantageous includes treatment of areas of thin skin, e.g. the skin around the eyes, where it would be more difficult to inject a needle. In such a scenario, the cannula may be inserted closer to the cheek and woven under the skin up to the thinned eye area. Use of a cannula may yield less pain during treatment as fewer needle sticks may be required, as well as potentially yielding a faster recovery time as the risk of bleeding and bruising is decreased due to the blunt needle having a lower chance of breaking vasculature during injection and/or repositioning. The use of a microcannula versus a needle reduces the risk of injecting filler into a blood vessel, as the blunt end of the cannula may simply push aside encountered vessels unlike the pointed end of a needle which is likely to puncture an encountered vessel. On the other hand, there are a number of circumstances where use of a needle is preferred over a cannula for administration of the formulations described herein (and such methods are typically more affordable). For such aesthetic treatments such as treatment of acne scars, use of a needle may be preferable medically and/or from the perspective of the recipient/subject, as would be the case for the treatment of lines being filled which are particularly superficial or shallow.

In addition to delivery systems, the invention also provides kits and packaged compositions that comprise separate components of the delivery systems described herein. Thus, in one aspect the invention provides a kit comprising (a) dried, solid composition of RPP particles (typically in combination with excipients, such as gel forming agents and, optionally, surfactants, buffers, and the like), usually in a sealed package or container, (b) a diluent, such as WFI, in a container that maintains the volume and prevents contamination, and (c) one or more parts of a delivery system including a storage container and a needle or other delivery component. The kit or package may include materials and/or instructions for reconstitution and application of the reconstituted product as well as patient information, instructions, or the like.

The kits and delivery systems can incorporate any features of the above-described compositions. In one exemplary aspect, the invention provides a kit comprising a) vial comprising lyophilized PLLA (optionally with one or more other excipients present, such as CMC and/or a surfactant, such as a polysorbate surfactant); and b) prefilled syringe comprising diluent, such as WFI. In some embodiments, the vial containing lyophilized PLLA may be hermetically sealed. In certain embodiments, the vial may be sealed using a seal penetrable by a needle. In some embodiments, the diluent contained within the syringe may comprise one or more of polysorbate or other nonionic surfactant, sodium carboxymethyl cellulose or HPMC, and further optionally disodium hydrogen phosphate dihydrate, citric acid anhydrous, sodium chloride, and/or sodium hydroxide. In some embodiments, the water for injection may be used to bring the volume up to 100%. In some embodiments, the prefilled syringe may also comprise a needle. In some embodiments, the kit may also or alternatively comprise an injection needle and a cannula for insertion as is known in the art using the injection needle as guidance. In certain embodiments, the kit may also comprise instructions for use of the product contained therein for the physician, patent, or any combination of the two.

In a preferred embodiment, the present invention is a kit comprising at least one vial comprising lyophilized powder having between about 50 mg and about 250 mg of PLLA, for example about 50 mg, 60 mg, 70 mg or 80 mg of PLLA, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, or about 250 mg of PLLA. In a more preferred embodiment, the vial may comprise approximately 100 mg to about 200 mg of PLLA. Preferably, the vial may contain about 150 mg of PLLA.

In a preferred embodiment, the kit of the present invention contains a prefilled syringe comprising diluent comprising polysorbate 80 and/or polysorbate 20 in an amount ranging from about 0.01% to about 0.30% w/v, for example about 0.02%, 0.03%, 0.04%, 0.05%, about 0.06%, 0.07%, 0.08%, 0.09%, 0.10%, 0.11%, 0.12%, 0.13%, 0.14%, 0.15%, 0.16%, 0.17%, 0.18%, 0.19% or about 0.20%, approximately 0.21%, 0.22%, 0.23%, 0.24%, 0.25%, 0.26%, 0.27%, 0.28%, 0.29% or about 0.30% w/v polysorbate 20. In a more preferred embodiment, the prefilled syringe comprises diluent having polysorbate 20 in an amount ranging from 0.05% to about 0.15%. Preferably, the vial may contain about 0.1% w/v polysorbate 20.

In a preferred embodiment, the kit of the present invention contains a prefilled syringe comprising diluent having sodium carboxymethyl cellulose in an amount ranging from about 1% to about 4% w/v, for example about 1%, about 1.1%, about 1.2%, about 1.3%, about 1.4%, approximately 1.5%, approximately 1.6% or about 1.7%, for example about 1.8%, about 1.9%, about 2.0%, about 2.1%, about 2.2% or about 2.3% w/v sodium carboxymethyl cellulose, e.g. about 2.4%, about 2.5%, about 2.6%, about 2.7%, about 2.8%, about 2.9%, about 3.0% or about 3.1%, for example about 3.2%, about 3.3%, about 3.4%, about 3.5%, about 3.6%, about 3.7%, about 3.8%, about 3.9% or about 4.0% sodium carboxymethyl cellulose w/v. In an embodiment, the prefilled syringe comprises diluent having sodium carboxymethyl cellulose in an amount ranging from about 1.5% to 3.5%. Preferably, the vial may contain about 2.3% w/v sodium carboxymethyl cellulose.

In certain embodiments, the kit of the present invention also comprises disodium hydrogen phosphate dihydrate (e.g., about 0.1-0.15%, such as about 0.13% w/v); citric acid anhydrous (e.g., about 0.05-0.15%, such as about 0.1% w/v); sodium chloride (e.g., about 0.25-about 0.75%, such as about 0.6% w/v); and/or sodium hydroxide (e.g., about 0.02-0.08%, such as about 0.05% w/v).

According to some facets, a kit of the present invention may comprise more than one vial, each comprising a single administration of a composition described herein. In certain facets, two or more applications or administrations of one or more compositions described herein may be prescribed and thus a plurality of vials within a kit may be utilized for such a purpose.

### Methods of Use

The invention also provides a number of novel methods of using RPP particles, related compositions, and related delivery systems, as described above and elsewhere herein.

In one exemplary aspect, the invention provides a method of delivering compositions of the invention, e.g., a neocollagenic dermatological implant/injection composition, to a mammalian recipient, such as a human aesthetic patient, comprising contacting the recipient with a needle delivery system as described above and applying a delivery force to the needle system to deliver an effective amount of the composition to the body of the recipient. In most aspects, the recipient will be a human aesthetic patient and the composition will be administered by or under the direction and/or supervision of a licensed healthcare provider, such as a licensed physician.

The RPP particle compositions when combined with the delivery systems provided herein offer high levels of delivery efficiency, especially when compared to SCULPTRA®. “Injection failure” means the occurrence of a blockage of a needle in the process of injecting an RPP particle composition. Systems of the invention provided herein (e.g., a needle system comprising a 26G or 27G needle in combination with PLLA microspheres having an average maximum size of 35-55 μm in at least about 90% of the particles) are associated with delivery failures of 40% or less. In some aspects, such delivery systems and RPP compositions of the invention are associated with injection failure rates of less than about 30%, less than about 25%, less than about 20%, less than about 15%, less than about 10%, or less than about 5%.

Because of the substantially smaller and more uniform nature of RPP particles in such systems, the force required to deliver a reconstituted or otherwise liquid RPP particle composition through a standard needle system (e.g., a conventional 26G needle system) will be significantly less in the inventive system than in SCULPTRA® systems.

Such force can be evaluated through the minimum force required to deliver the composition, the maximum force required to deliver the composition, and mean force used to deliver the composition through the needle delivery system. In one aspect, the minimum force used to deliver the composition through the needle of the system is about 40 to about 80 g. In another facet, the minimum force is about 40-55 g, such as about 50 g.

In another aspect, the maximum force used to deliver the composition is also or alternatively about 100 g-about 2500 g, such as about 175 g-about 1750 g. In still more specific aspects, the maximum force is about 200 g-about 1200 g, such as about 200 g-about 700 g.

In still another embodiment, the mean force used to deliver the composition through the needle system is about 100 g-about 1000 g. In more specific aspects, the mean force used to deliver the composition is about 150 g-750 g, such as about 175 g-about 400 g, or, more precisely still, about 200 g-about 350 g. According to certain aspects, the mean force used to delivery a composition described herein through a given needle delivery system is significantly less than that required to delivery a composition of a comparable on market product such as SCULPTRA® through the same sized (e.g., same needle inner diameter) needle delivery system, such as for example, such a mean force is at least 1%, at least 3%, at least 5%, at least 8%, at least 10%, at least 15%, or even at least 20% or more less than the mean force required for a comparison product.

The measurement of such forces can be performed by any suitable manner and can apply to a number of systems or an individual system. In one aspect, such forces are measured in accordance with ISO11040-4 section E (glide force test method to evaluate syringe lubrication). Devices that can be used for carrying out such measurements are available from Texture Technologies in the USA (see, e.g., http://texturetechnologies.com/application-studiesisyringability).

Typically the delivery systems of the invention deliver compositions of the invention into the body of a mammalian recipient so as to induce a physiological effect, such as detectably and usually significantly measurably increasing the volume of the skin of the recipient (e.g., to a level at least equivalent to that observed in treatment with SCULPTRA®). Thus, the invention provides a method of modifying the skin or the appearance of the skin of a human patient comprising subcutaneous or intradermal injection or implantation of an RPP particle composition of the invention. Such methods can be used to promote or carry out reparative or plastic surgery, or for filling wrinkles, fine lines, skin cracks, acne scars and other scars, as well as in dentistry for filling the gums, and for other applications associated with SCULPTRA®.

Age related skin sagging and wrinkling with age is a result of the body's slowed production of, and decreasing density of, collagen and elastin which provide key structural integrity of skin. In the aesthetic treatment of such sagging or wrinkling, dermal fillers and/or RPP compositions can be used. The RPP particles of the invention, such as PLLA monospheres of the inventive formulations provided herein, are biocompatible, biodegradable polymers which stimulate the process of neocollagenesis, or the body's own production of new collagen, in the areas where the formulation is injected. Such neogenesis results in a more permanent aesthetic solution compared to dermal fillers which, upon degradation, eliminate the aesthetic improvement provided by their presence. In some cases, however, it may take about 2-8, such as about 3-6 months, for the effects of treatment with the RPP particle compositions of the invention to be seen or at least to reach near maximum effect.

Use of the systems and products of the invention can comprise repeat administration of such compositions as part of a course of treatment. Thus, for example, a course of treatment can comprise two, three, four, or more administrations of product, which may be separated by one, two, three, or four weeks, or about 10, about 20, about 25, or about 30 days (e.g., about 2-7 weeks, such as about every 3-6 weeks). Each treatment session may include administration of product into multiple sites, which may or may not be associated with a single area of the body, such as a single area of the skin (e.g., the nasolabial fold). The amount of a reconstituted/liquid product delivered in a single injection typically will be about 1.5 to about 5 mL, such as about 2 to about 4 mL, e.g., about 2.5 or about 3 mL. In some cases, injection is performed in a grid around a target site. In some aspects, the injection is primarily or entirely delivered into the dermis. It also may be the case that this can reflect the amount used in a single session, which may be injected into one or more locations in that treatment session.

In some aspects, the delivery of the composition of the invention is performed association with the administration of an effective amount of an anesthetic. In one aspect, the anesthetic is a component of the formulation (e.g., is added with the diluent at reconstitution). In another aspect, the anesthetic is co-administered or administered in association with the injection/implantation of the RPP particle administration (e.g., is administered before injection).

In still another aspect, the method is applied as an aesthetic treatment in combination with one or more other aesthetic modification methods, which can include other surgical techniques, application of other dermal fillers, and/or application of one or more pharmaceutical products or medical devices. Aesthetic products which modify skin aesthetics via modes other than via neocollagenesis may be synergistic. Such treatments may include but not be limited to dermal fillers (e.g. Restylane®, Juvederm®, Restylane® Lyft (formerly Perlane®), or other similar dermal fillers providing bulk to targeted skin areas), and neuromodulators (e.g. abobotulinumtoxinA (Dysport®), onabotulinumtoxinA (Botox®) or other similar aesthetic treatments which serve to modify skin aesthetics through modification of nerve inputs). Other treatments which may be used synergistically are neocollagenic treatments using radio frequency or light frequency or pulsed light to stimulate collagen growth (e.g. ReFirme™, FotoFacial®, or similar such products.) Because the neocollagenic dermatological implant described here works by stimulating collagen production, it may take time to see visible results. As such, it may be beneficial to utilize a volumizing dermal filler, as an example but not limited to a calcium-based microsphere technology such as exemplified by Radiesse® or a hyaluronic acid filler (e.g., Juvederm or Restylane) to provide short-term results while the dermatological implant of the current invention begins to stimulate collagen production. Such dermal fillers may provide an immediate aesthetic improvement while the implant of the current invention takes effect, and because they have a shorter lifespan, disappear as the effects of the neocollagenic dermatological implant of the current invention begin to appear.

In a particular aspect, the method of application includes administration of the product to patients with relatively thin skin. The subject may have, for example, skin in a target area that is at least about 15%, at least about 25%, or at least about 30% thinner than average in the population. For example, the subject may be a patient that has or is undergoing a skin atrophy condition (e.g., corticosteroid skin atrophy) or has another condition associated with abnormally thin skin (e.g., anetoderma, vermiculate atrophoderma, or rheumatoid arthritis-associated skin thinning).

In other aspects, the method is performed in an area that is associated with fat loss, such as an area associated with facial lipoatrophy. Thus, the method can comprise restoration and/or correction of the signs of facial fat loss.

The compositions and methods of the invention also or alternatively are associated with lower rate of nodule formation than SCULPTRA®. According to embodiments the amount of nodule formulation associated with the methods and compositions of the invention is reduced by at least about 5%, at least about 10%, at least about 15%, at least about 20%, at least about 25%, or more, as compared with SCULPTRA® (e.g., as may be determined by comparison of clinical data or direct head-to-head studies of the products).

Also or alternatively, the product can be delivered to an area that is associated with thinner skin even in typical populations. In one aspect, the method comprises administering the composition to an area of a human face having an average epidermal thickness of less than 40 μm, an average dermal thickness of less than 1200 μm, or both. In another aspect, the method is performed in area of the skin having an average epidermal thickness of less than 40.75 μm, an average dermal thickness of less than 1230 μm, or both. In still other aspects, the average epidermal thickness in the treated area is less than 40.5 μm, the average dermal thickness is less than about 1200 μm, or both are true.

In another aspect, the method comprises administering the composition to an area of a human face having an average epidermal thickness of less than 40 μm, an average dermal thickness of less than 1175 μm, or both.

In still another exemplary facet, the method comprises administering the composition to an area of human skin having an average relative thickness score of less than 1.5. In a more particular aspect, the area has an average relative thickness score of less than 1.35. Relative thickness is described in Chopra et al., Aesthetic Surgery Journal 2015, Vol 35(8) 1007-1013. Although such methods of assessing thin skin are exemplified herein that the scope of the invention is not limited to those patient types described in the Chopra article, but, rather, can be applied to subjects of different ages, races, conditions, etc. In a more general sense, one aspect of the invention comprises administering a composition of the invention to an area of a subject associated with skin that is at least about 5%, at least about 10%, at least about 12.5%, at least about 15%, at least about 17.5%, at least about 20%, at least about 25%, or at least about 30% thinner than the average skin of a subject of the same age or age group, sex, race, and/or condition.

In still another exemplary aspect, the method comprises administering the composition to a target area of human skin in a part of the body, such as the face and neck, wherein the average skin thickness in the target area (of a person or a group of persons) is at least about 10% less, at least about 15% less, at least about 20% less, or at least about 30% less than either (a) the average skin thickness in the person (or average person of such an age, gender, sex, and/or condition) or (b) the part of the body in the person (or an average of a group of similar person) that is associated with the thickest skin in the person (or population). This is another aspect reflecting that the method can be practiced on areas of relatively thin skin in patients.

### EXPERIMENTAL DATA (EXAMPLES)

The following experimental data (examples) support and exemplify various aspects of the invention. This disclosure is provided to further illustrate the principles of the invention and should be not be interpreted as limiting its scope in any manner.

### Materials

PLLA particles were produced according to the particle production methods described in WO200511559A1 enabling the production of particles in a monodisperse manner. The method was performed with the intent of producing particles with an average size of about 35 μm, with 95% of the particles having a size between 25 μm-45 μm. SCULPTRA® (batch number A7123) was purchased through Walter Krebs.

### Example 1

The following experiment was conducted to demonstrate that monodisperse microspheres, or monospheres, of an RPP composition, particularly PLLA, lead to less needle blockage and improved product injectability over the leading commercial conventional polydisperse PLLA composition in SCULPTRA®.

### Sample Preparation

Reconstitution was performed following the SCULPTRA® Brazilian product label (reconstitution by hydration with WFI for 2 hours). This resulted in a PLLA suspension density of 18.75 mg/mL at room temperature (RT). From the resulting homogenized suspension, 7 syringes (Becton Dickinson, 1 mL disposable with luer lock, not included in the SCULPTRA® package) were filled with 1 mL in order to analyse the injectability of SCULPTRA® in 7-fold. Needles 26G, ⅝″ from Becton Dickinson (Microlance 3, not included in the SCULPTRA® package) were used.

Monodisperse PLLA microspheres were prepared by dissolving PLLA in dichloromethane (DCM), 10-15 wt %. The organic phase was passed through a proprietary microsieve (Nanomi) into the aqueous phase and the resulting suspension was allowed to extract for 3 to 24 hours. The microspheres were collected and sieved using 75 and 10 μm sieves. Drying of the microspheres occurred either via freeze drying or using a nitrogen flow.

Monodisperse PLLA monospheres (Nanomi, test material, 2087-EKK-04Oct2018-A) were reconstituted by addition of 7.5 mL of diluent to 150 mg of test PLLA monosphere material, resulting in a suspension density of 20 mg/mL at RT. The resulting suspension was kept at RT for 2 hours. From the suspension, 6 syringes were filled with 1 mL in order to analyse Nanomi's monospheres in 6-fold. The deviating volume of diluent added to the microspheres is directed by Nanomi's intention to include prefilled syringes which are currently produced in a lead product. These prefilled syringes each contain 2.5 mL of diluent.

### Injection Testing Analysis

Texture analyzer TA.XTplus (Stable Micro Systems—Surrey, UK (see https://www.stablemicrosystems.com/TAXTplus.html)) was used for this experiment. The injections were performed according to ISO11040-4 section E (glide force test method to evaluate syringe lubrication). In short, the syringes were placed into the texture analyser in a vertical position and ejected using a speed of 100 mm/min. The measured distance was 45 mm.

### Results

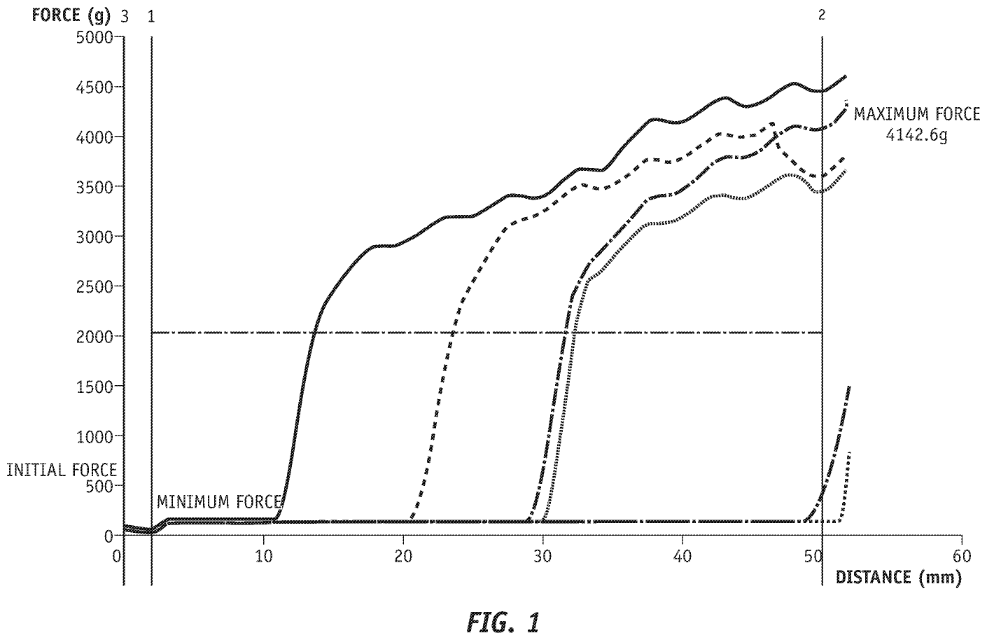

FIG. 1 is an injectability graph of SCULPTRA® samples using a 26G ⅝″ needle. The first interval in FIG. 1 (space between vertical lines 3 to 1) in the graph shows the force required to fill the needle with the suspension. During the second interval (1 to 2), the injectability is tested. The results of this interval are reported in Table 2 below. Each curve in the graph of FIG. 1 represents an individual injection.

The data obtained for the analysis of SCULPTRA® is also presented in Table 1, below.

TABLE 1
Injectability results for SCULPTRA ® with a 26 G ⅝″ needle

| Repetition no. | Mean force (g) | Min. force (g) | Max force (g) |
|---|---|---|---|
| *1 | 2050 | 64 | 4143 |
| 2 | 151 | 78 | 401 |
| *3 | 1458 | 58 | 4108 |
| *4 | 2822 | 78 | 4533 |
| *5 | 1305 | 57 | 3619 |
| 6 | 158 | 54 | 177 |
| 7 | 137 | 41 | 159 |
| Average | 1154 | 61 | 2448 |
| S.D. | 1059 | 13 | 2079 |

The asterisks shown in Table 1 for repetition numbers 1, 3, 4 and 5, and the corresponding injectability graphs for each of repetitions 1, 3, 4, and 5, shown in FIG. 1, mark repetitions wherein the needle became blocked during injection.

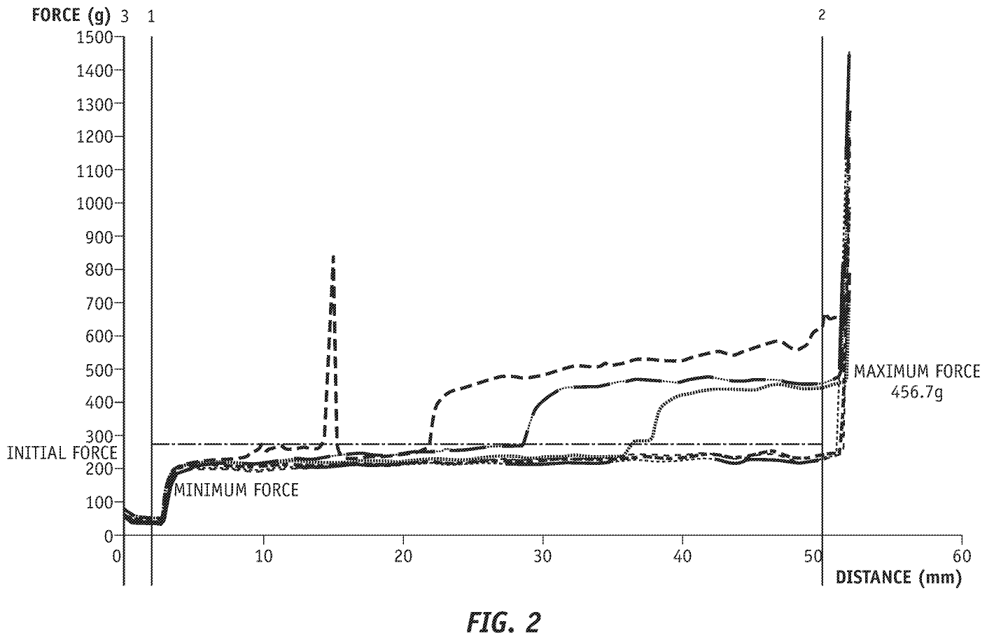

FIG. 2 shows the injectability results for Nanomi monodisperse PLLA microsphere test formulations injected with a 26G ⅝″ needle. The first interval in FIG. 2 (3 to 1) in the graph shows the force required to fill the needle with the suspension. During the second interval (1 to 2), the injectability is tested. The results of this interval are reported in Table 2, below. Each curve represents an individual injection. This data reflects a failure rate of 4/7 for delivery of SCULPTRA® through such needles (about 57%).

The data obtained for the analysis of Nanomi monodisperse PLLA microsphere test formulations is also presented in Table 2, below, and are graphically presented in FIG. 2.

TABLE 2
Injectability results for Monodisperse Microspheres
with a 26 G ⅝″ needle

| Repetition no. | Mean force (g) | Min. force (g) | Max force (g) |
|---|---|---|---|
| 1 | 212 | 44 | 234 |
| 2 | 218 | 35 | 251 |
| 3 | 404 | 45 | 842 |
| 4 | 330 | 43 | 479 |
| 5 | 222 | 47 | 256 |
| 6 | 278 | 52 | 457 |
| Average | 277 | 44 | 420 |
| S.D. | 77 | 5 | 234 |

The data illustrated in FIGS. 1 and 2, and presented in Tables 1 and 2, above, show that the exemplary monodisperse PLLA microspheres of the invention have significantly different properties in terms of injection force requirements as compared with SCULPTRA®. E.g., none of tested monodisperse PLLA microsphere compositions required the average force of 2000 observed with SCULPTRA®, in fact all required about 25% or less force than the ˜2000 required for SCULPTRA®, and several required even less force. These types of lower force requirements associated with microparticle compositions of the invention represent another aspect of the invention.

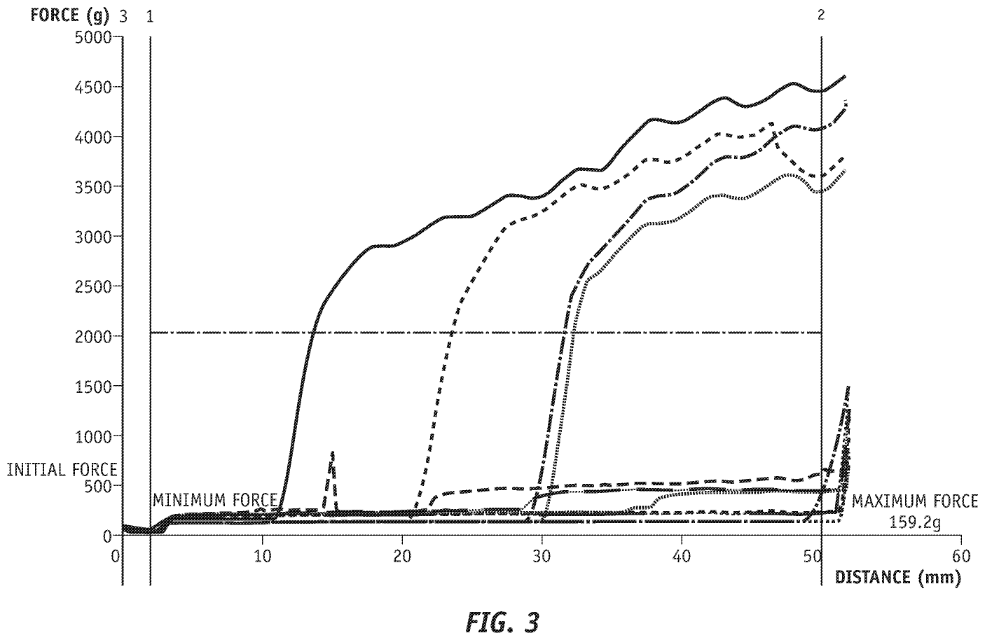

FIG. 3 is an overlay of the injectability measurements of SCULPTRA® vs the monodisperse microspheres as tested with a 26G ⅝″ needle.

From the results shown in in FIGS. 1-3 and Tables 1 and 2, reconstitution following the Brazilian label instructions for SCULPTRA® led to formulation conditions where the product did not pass the 26G needle without issues, specifically as four out of seven injections became blocked. It may be noted that the European label for SCULPTRA® recommends a lower reconstitution volume, yielding a more concentrated final product. It is possible that such an increase in concentration may yield an even higher possibility of needle blockage. While it may be possible that this blockage could be overcome through the use of a larger needle size, needles with a larger diameter with larger diameters would not be favored in aesthetic medicine, as they may increase patient discomfort.

The results obtained for the injectability of Monodisperse microspheres proves that these RPP particles passed through the selected 26G needle with much lower mean force (24%) max force (17%) and with much less variability as compared to SCULPTRA® (see, e.g., FIG. 3). Repetition 3 (long dashed line) of FIG. 2 showing the injectability of monodisperse microspheres shows a small spike in the maximum force required for dispensing (842 g) over the course of approximately 15 mm represents only about ⅓ of the average maximum force observed with SCULPTRA®. This did not result in obstruction of the needle and the measurement continued without operator interference. It is evident much more force needs to be applied when injecting SCULPTRA® compared to the Monodisperse microspheres.

### Example 2

This Example reports on the analysis of particle size and distribution of both SCULPTRA® and the PLLA monospheres of this exemplary aspect of the invention. For this measurement, a Multisizer™ 3 Coulter Counter was used, purchased from Beckman. The PLLA particles were resuspended in water and measured using Coulter® Isoton® II diluent.

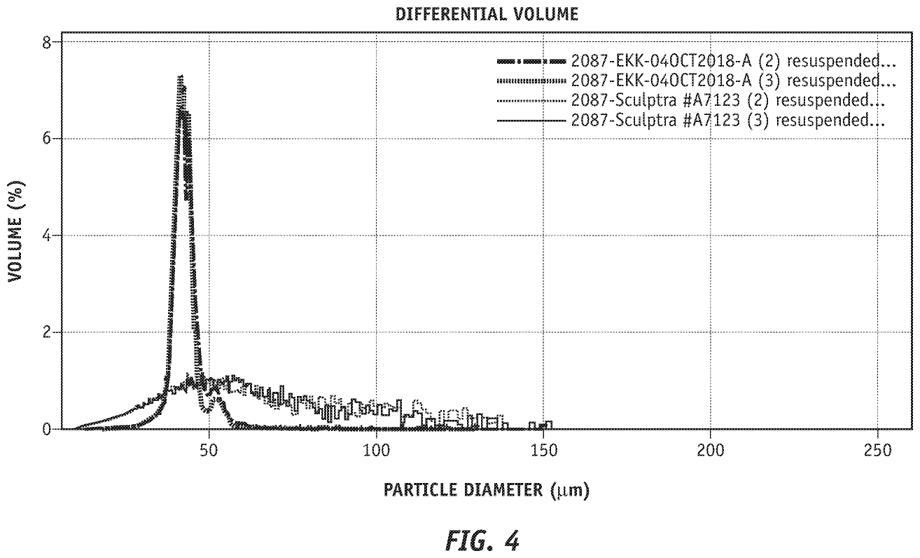

As reported above, SCULPTRA® PLLA particles are polydisperse, including a range of particle sizes. However, monodisperse microspheres of invention are typically monodisperse with a narrow size distribution (e.g., at least about 90% of the particles have a maximum diameter and/or average diameter of within about 10 μm of each other). FIG. 4 shows a comparison between SCULPTRA® particles (samples labeled as “2087-sculptra . . . ”) and the microspheres of the inventive monodisperse PLLA microsphere formulation (samples labeled as “2087-EKK . . . ”).

As evident in FIG. 4, The particles in SCULPTRA® are varied in size, having detected sizes between 0 and 150 μm. On the other hand, the monodisperse monospheres formulation contains polymeric particles which fall within a narrow range of 25 to 70 μm, with most particles falling within the range of 35-45 μm.

Typically, a 26G needle has an inner diameter of 260 μm. When in the case of monodisperse monospheres, three particles of even 70 μm agglomerate, the 26G needle will not be blocked, as an agglomeration of approximately 210 μm is created, that being still less than the 260 μm inner diameter of the 26G needle (the same would be true for four particles of about 50 μm in maximum diameter). However, when in case of SCULPTRA® three particles of 150 μm agglomerate, the risk of blockage of the needle is quite high as that creates an agglomeration of approximately 450 μm, well above the size of the 260 μm inner diameter of a 26G needle.

It appears therefore clear that an even smaller sized needle than the 26G needled utilized in this Example could be utilized for injecting a dermal filler utilizing the inventive PLLA monospheres tested herein, leading to increased patient comfort, patient compliance, and less waste of product.

### Example 3

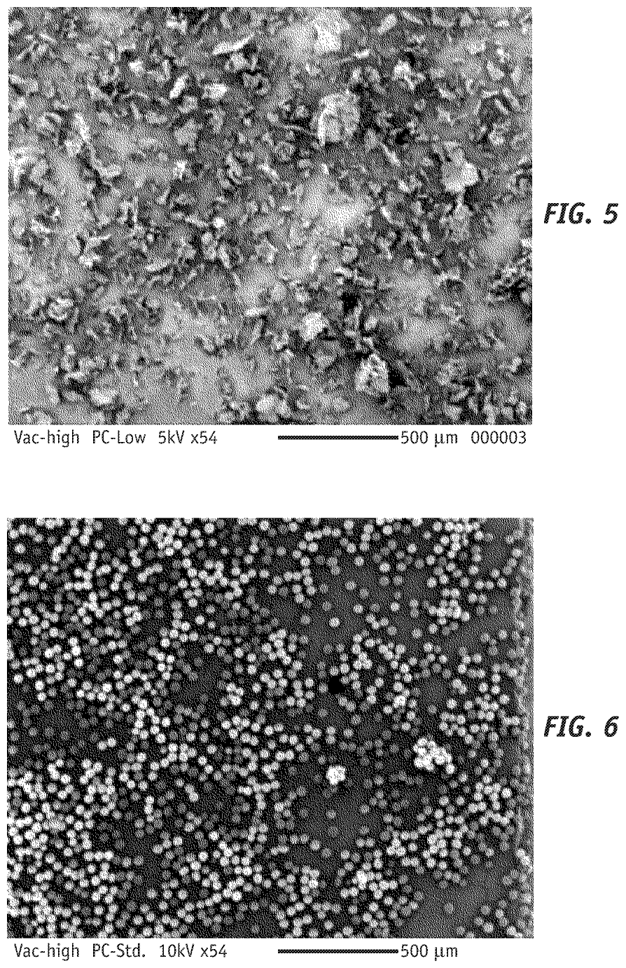

SCULPTRA® particles and the PLLA monospheres of the invention described above were examined using scanning electron microscopy (SEM) purchased from JEOL. Exemplary scans are shown in FIG. 5 (SCULPTRA®) and FIG. 6 (PLLA monospheres).

As evident from FIGS. 5 and 6, evaluation of both products using SEM shows a very distinct difference between the microspheres in SCULPTRA® vs. the inventive PLLA monospheres. The RPP particles of SCULPTRA® contain crude and sharp-edged material. Such rough, inconsistent shaping enhances agglomeration. On the other hand, the inventive PLLA monospheres are round, even, and consistent, enabling them to pass through a 26G needle in a smooth manner.

### Example 4

Three separately manufactured RPP particle formulations were subjected to an industry standard accelerated stability conditions stability testing protocol. Initial results, reported as “initial value,” were obtained after production of each batch. Stability data, reported as “t=0 months” and “t=6 months @ 40° C./75% RH”, were collected at the beginning of the stability study (time point 0) and after six months of storage under accelerated stability conditions of 40° C. and 75% relative humidity.

The stability protocol measured molecular weight distribution (molecular weight and polydispersity index (PDI)) and crystallinity in two samples from each production batch after initial production, at the beginning of the stability study, and at the six-month time point.

Particle size distribution data was collected as single average for each batch at the beginning of the stability study and at the six-month time point. Data for each of the 3 batches are presented in Tables 3-5, respectively:

TABLE 3
T = 0 and T = 6 mo. Stability Data for 2087-DBE-13MAR19-A04
2087-DBE-13MAR19-A04

| | | |
|---|---|---|
| Sample 1 | Sample 2 | Average |

Molecular Weight Distribution

| | | | |
|---|---|---|---|
| Molecular Weight (Mw) in kDa | | | |
| Initial value: | 110.7 | 111.8 | 113.3 |
| t = 0 months: | 112.8 | 112.6 | 112.7 |
| t = 6 months @ 40° C./75% RH: | 112.5 | 112.4 | 112.5 |
| PDI (Mw/Mn) | | | |
| Initial value: | 1.37 | 1.38 | 1.38 |
| t = 0 months: | 1.40 | 1.38 | 1.39 |
| t = 6 months @ 40° C./75% RH: | 1.39 | 1.37 | 1.38 |

Crystallinity

| | | | |
|---|---|---|---|
| Initial value: | 0 | 0 | 0.0 |
| t = 0 months: | 0 | 0 | 0.0 |
| t = 6 months @ 40° C./75% RH: | 0 | 0 | 0.0 |

Particle Size Distribution

| | |
|---|---|
| d10 (μm) | |
| Initial value: | — |
| t = 0 months: | 27.19 |
| t = 6 months @ 40° C./75% RH: | 27.56 |
| d50 (μm) | |
| Initial value: | — |
| t = 0 months: | 29.17 |
| t = 6 months @ 40° C./75% RH: | 30.19 |
| d90 (μm) | |
| Initial value: | — |
| t = 0 months: | 31.37 |
| t = 6 months @ 40° C./75% RH: | 76.17 |
| Mean (μm) | |
| Initial value: | — |
| t = 0 months: | 29.71 |
| t = 6 months @ 40° C./75% RH: | 42.12 |

TABLE 4
T = 0 and T = 6 mo. Stability Data for 2087-DBE-03APR19-A02
2087-DBE-03APR19-A02

| | | |
|---|---|---|
| Sample 1 | Sample 2 | Average |

Molecular Weight Distribution

| | | | |
|---|---|---|---|
| Molecular Weight (Mw) in kDa | | | |
| Initial value: | 110.7 | 111.8 | 111.3 |
| t = 0 months: | 112 | 112.1 | 112.1 |
| t = 6 months @ 40° C./75% RH: | 113.2 | 112.7 | 113.0 |
| PDI (Mw/Mn) | | | |
| Initial value: | 1.37 | 1.38 | 1.38 |
| t = 0 months: | 1.37 | 1.36 | 1.37 |
| t = 6 months @ 40° C./75% RH: | 1.37 | 1.37 | 1.37 |

Crystallinity

| | | | |
|---|---|---|---|
| Initial value: | 5.3 | 5.3 | 5.3 |
| t = 0 months: | 5.3 | 5.3 | 5.3 |
| t = 6 months @ 40° C./75% RH: | 4.7 | 4.7 | 4.7 |

Particle Size Distribution

| | |
|---|---|
| d10 (μm) | |
| Initial value: | — |
| t = 0 months: | 28.84 |
| t = 6 months @ 40° C./75% RH: | 28.71 |
| d50 (μm) | |
| Initial value: | — |
| t = 0 months: | 31.93 |
| t = 6 months @ 40° C./75% RH: | 31.88 |
| d90 (μm) | |
| Initial value: | — |
| t = 0 months: | 73.21 |
| t = 6 months @ 40° C./75% RH: | 73.81 |
| Mean (μm) | |
| Initial value: | — |
| t = 0 months: | 39.26 |
| t = 6 months @ 40° C./75% RH: | 39.24 |

TABLE 5
T = 0 and T = 6 mo. Stability Data for 2087-DBE-06MAR19-A01
2087-DBE-06MAR19-A0

| | | |
|---|---|---|
| Sample 1 | Sample 2 | Average |

Molecular Weight Distribution

| | | | |
|---|---|---|---|
| Molecular Weight (Mw) in kDa | | | |
| Initial value: | 108.2 | 107.4 | 107.8 |
| t = 0 months: | 108.7 | 109.3 | 109.0 |
| t = 6 months @ 40° C./75% RH: | 109 | 108.9 | 109.0 |
| PDI (Mw/Mn) | | | |
| Initial value: | 1.29 | 1.30 | 1.30 |
| t = 0 months: | 1.32 | 1.31 | 1.32 |
| t = 6 months @ 40° C./75% RH: | 1.32 | 1.33 | 1.33 |

Crystallinity

| | | | |
|---|---|---|---|
| Initial value: | 10.7 | 10.0 | 10.4 |
| t = 0 months: | 9.6 | 10.2 | 9.9 |
| t = 6 months @ 40° C./75% RH: | 9.7 | 9.3 | 9.5 |

Particle Size Distribution

| | |
|---|---|
| d10 (μm) | |
| Initial value: | — |
| t = 0 months: | 28.07 |
| t = months @ 40° C./75% RH: | 28.01 |
| d50 (μm) | |
| Initial value: | — |
| t = 0 months: | 29.57 |
| t = 6 months @ 40° C./75% RH: | 29.52 |
| d90 (μm) | |
| Initial value: | — |
| t = 0 months: | 31.44 |
| t = 6 months @ 40° C./75% RH: | 31.66 |
| Mean (μm) | |
| Initial value: | — |
| t = 0 months: | 29.96 |
| t = 6 months @ 40° C./75% RH: | 30.72 |

Data for the first batch, 2087-DBE-13MAR19-A04 (Table 3) show that the average molecular weight in kDa of the particles varied by approximately 0.7% from the initial value to the value measured at the 6-month stability study time point. The polydispersity index (PDI), a value indicating the breadth of the molecular weight distribution of the particles, varied by about 0.7%. Particle size distribution, reported for d10, d50, d90, and mean particle size in μm with values indicating the percentage of particles smaller than the indicated d-number (e.g. a d10 value of 30 would indicate that 30% of the particles are smaller than 10 μm and 70% are larger than 10 μm) indicated a variance from time point 0 of the stability study to the 6 month time point of the stability study of approximately 1.3% for d10, 3.4% for d50, 58.8% for d90, and 29.5% for the mean. It is worthy of note that such data indicate that the particles of the formulation are not significantly decreasing in volume over time.

Data for the second batch, 2087-DBE-03APR19-A02 (Table 4) show that the average molecular weight of the particles varied by less than 0.1% from initial measurement to the 6-month stability time point. The PDI varied by about 0.7%. Degree of crystallinity was measured and is also presented for this batch. Crystallinity varied by about 11.3% over the same period. Particle size distribution variance was reported at approximately 0.5% for d10, 0.2% for d50, 0.8% for d90, and less than 0.1% for the mean between time point zero of the stability study and the 6-month stability time point.

Data for the third batch, 2087-DBE-06MAR19-A01 (Table 5) show that the average molecular weight of the particles varied by approximately 1.1%, PDI varied by approximately 2.26%, and crystallinity varied by approximately 8.7% from initial measurement to the 6 month stability time point. Particle size distribution variance was reported at approximately 0.2% for d10, 0.2% for d50, 0.7% for d90, and 2.5% for the mean between time point zero of the stability study and the 6-month stability time point.

These data demonstrate that particles of the invention exhibit favorable stability characteristics over sustained periods of time. For example, mean particle sizes did not decrease significantly over the course of the accelerated stability testing study conducted, and in most cases any variation in size was within 10% or less, such as within 5% or within 3% or less. These and other data reflect stability and other beneficial properties associated with the microparticle formulations of the invention.

### Exemplary Aspects of the Invention

The following is a non-limiting list of exemplary aspects of the invention, which is intended to highlight some of the various embodiments of the invention:

1. An implant/injection composition comprising an effective amount of particles that are at least primarily composed of one or more resorbable polyester polymers and that have a size distribution such that at least about 70% of the particles have a maximum particle diameter that is within about 30% of the average particle diameter of the particles in the composition;
2. The implant/injection composition of Aspect 1, wherein the particles consist essentially of one or more resorbable polyester polymers;
3. The implant/injection composition of Aspect 1 or Aspect 2, wherein at least about 80% of the particles have a maximum particle diameter that is within 25% of the mean particle diameter of the particles in the composition;
4. The implant/injection composition of Aspect 3, wherein at least about 85% of the particles of the composition have a maximum particle diameter that is within 20% of the mean particle diameter of the particles in the composition;
5. The injection/implant composition of Aspect 4, wherein at least about 90% of the particles of the composition have a maximum particle diameter that is within 18% of the mean particle diameter of the particles in the composition;
6. The injection/implant composition of any one of Aspects 1-5, wherein at least about 75% of the particles of the composition have a maximum particle diameter that is within 15% of the mean particle diameter of the particles in the composition;
7. The injection/implant composition of any one of Aspects 1-6, wherein at least a majority of the one or more polyester polymers are neocollagenic, such that the composition is detectably neocollagenic when administered to the skin of a human patient;
8. The injection/implant composition of any one of Aspects 1-7, wherein the one or more polyester polymers are derived primarily from starting materials obtained from plants;
9. The injection/implant composition of any one of Aspects 1-7, wherein the one or more polyester polymers are produced through chemical synthesis;
10. The injection/implant composition of Aspect 8 or Aspect 9, wherein the one or more polyester polymers are at least primarily composed of a polyglycolide (a PGA), a polylactic acid (a PLA), a polycaprolactone (a PCL), a polyhydroxybutyrate (a PHB), or a mixture of two or more thereof;
11. The injection/implant composition of Aspect 10, wherein the one or more polyester polymers consist essentially of a PGA, a PLA, a PCL, a PHB, a copolymer of two or more thereof, or a mixture of any two or more thereof;
12. The injection/implant composition of Aspect 11, wherein the one or more polyester polymers consist of a PGA, a PLA, a PCL, a PHB, a copolymer of two or more thereof, or a mixture of any two or more thereof;
13. The injection/implant composition of Aspect 12, wherein the one or more polyester polymers consist essentially of polylactic-co-glycolic acid (PLGA), a PLA, or a mixture thereof;
14. The injection/implant composition of any one of Aspects 10-13, wherein at least 50% of the polyester polymer content of the composition is composed of a PLA;
15. The injection/implant composition of any one of Aspects 10-14, wherein at least 50% of the particles consist essentially of a PLA;
16. The injection/implant composition of Aspect 15, wherein the PLA is poly-L-lactic acid (PLLA) (crystalline), poly-D-lactic acid (PDLA) (crystalline), poly-D,L-lactic acid (PDLLA) (amorphous), or a mixture thereof;
17. The injection/implant composition of Aspect 16, wherein at least 50% of the polyester polymer content of the composition is composed of PLLA;
18. The injection/implant composition of Aspect 16 or Aspect 17, wherein the at least about 70% of the particles consist essentially of PLLA;
19. The injection/implant composition of any one of Aspects 10-18, wherein the filler particle polymer has a molecular mass of between 10,000 and 650,000 Daltons;
20. The injection/implant composition of Aspect 19, wherein the filler particle polymer has a molecular mass of between 50,000 and 250,000 Daltons;
21. The injection/implant composition of Aspect 19, wherein the filler particle polymer has a molecular mass of between 90,000 and 110,000 Daltons;
22. The injection/implant composition of any one of Aspects 10-21, wherein the inherent viscosity of the filler particle polymer is between 0.1-4.0 dL/g;
23. The injection/implant composition of Aspect 22, wherein the inherent viscosity of the filler particle polymer is between 0.40 and 2.0 dL/g;
24. The injection/implant composition of Aspect 23, wherein the inherent viscosity of the filler particle polymer is between 0.90 and 1.10 dL/g;
25. The injection/implant composition according to any one of Aspects 10-24, wherein the specific rotation of the filler particle polymer is between about −150 and about −160 degrees;
26. The injection/implant composition according to any one of Aspects 10-25, wherein the filler particle polymer has a melting point of between about 100 and about 300 degrees Celsius;
27. The injection/implant composition of Aspect 26, wherein the filler particle has a melting point of about 150-about 250 degrees Celsius;
28. The injection/implant composition of Aspect 27, wherein the filler particle has a melting point of about 175-about 195 degrees Celsius;
29. The injection/implant composition according to any one of Aspects 10-28, wherein the filler particle polymer has a heat of fusion of between about 25 J/g and about 150 J/g;
30. The injection/implant composition according to any one of Aspects 29, wherein the filler particle polymer has a heat of fusion of between about 75 J/g and about 125 J/g;
31. The injection/implant composition according to any one of Aspects 29, wherein the filler particle polymer has a heat of fusion of between about 85 J/g and about 95 J/g;
32. The injection/implant composition according to any one of Aspects 10-31, wherein the filler particle polymer has a quantity of residual solvents of less than about 0.01%;
33. The injection/implant composition according to any one of Aspects 10-32, wherein the filler particle polymer has a proportion of residual monomer (e.g., lactic acid) of less than about 0.1%;
34. The injection/implant composition according to any one of Aspects 1-33, wherein the maximum average diameter or dimension in any direction of three agglomerated particles of the composition is less than 250 μm;
35. The injection/implant composition of any one of Aspects 1-34, wherein in at least about 80% of the particles the diameter in any direction varies by no more than about 25%;
36. The injection/implant composition of Aspect 35, wherein in at least about 90% of the particles the diameter in any direction varies by no more than about 15%;
37. The injection/implant composition according to any one of Aspects 1-36, wherein the particles are microspheres in which at least 90% of the microspheres have a maximum diameter within a range of 30 μm (+/−12.5 μm from the average particle diameter);
38. The injection/implant composition according to any one of Aspects 1-37, wherein the injection/implant results in a measurable increase in dermal volume for an average period of at least about 2 years in subjects that receive one or more treatments comprising implanting an effective amount of implant/injection;
39. The injection/implant composition according to any one of Aspects 1-38, wherein less than 10% of the particles have a maximum diameter of less than 5 μm;
40. The injection/implant composition according to any one of Aspects 39, wherein less than 5% of the particles have a maximum diameter of less than 6 μm;
41. The injection/implant composition according to Aspect 39, wherein less than 2% of the particles have a maximum diameter of less than 9 μm;
42. The injection/implant composition according to any one of Aspects 1-41, wherein at least about 95% of the particles in the composition have a coefficient of variation of 50% or less when the average particle size is between 5 to 250 μm;
43. The injection/implant composition according to Aspect 42, wherein at least 95% of the particles in the composition have a coefficient of variation of 25% or less when the average particle size is between 5 to 250 μm;
44. The injection/implant composition according to Aspect 42, wherein in more than 95% of the particles in the composition are comprised with a coefficient of variation of maximum 15% measured over a range from 5 to 250 μm;
45. The injection/implant composition according to Aspect 42 or Aspect 43 or Aspect 44, wherein the number of particles that have a maximum diameter that is within 25% of the average maximum diameter is at least two times as great as the number of particles that differ from the average maximum diameter by 200% or more;
46. The injection/implant composition according to Aspect 45, wherein the number of particles that have a maximum diameter that is within 15% of the average maximum diameter is at least 2.5 times as great as the number of particles that differ from the average maximum diameter by 250% or more;
47. The injection/implant composition according to any one of Aspects 1-46, wherein for at least 95% of the particles in the composition the maximum size of an agglomeration of three injection/implant particles is less than 250 μm;
48. The injection/implant composition according to any one of Aspects 1-47, wherein for at least 97.5% of the particles in the composition the maximum size of an agglomeration of three injection/implant particles is less than 200 μm;
49. An injection/implant composition comprising (a) a filler component that is composed of an effective amount of particles that consist essentially of poly-L-lactic acid (PLLA) and (b) a physiological suitable carrier, wherein at least about 70% of the particles of the composition have a maximum diameter that is about 22.5 μm to about 62.5 μm in size;
50. The injection/implant composition of Aspect 49, wherein at least about 80% of the particles of the composition have a maximum particle diameter that is about 27.5 μm to about 60 μm in size;
51. The injection/implant composition of Aspect 50, wherein at least about 65% of the particles of the composition have a maximum particle diameter that is about 30 μm to about 50 μm in size;
52. The injection/implant composition of any one of Aspects 49-51, wherein in at least about 80% of the particles the diameter in any direction varies by no more than about 25%;
53. The injection/implant composition of Aspect 52, wherein in at least about 90% of the particles the diameter in any direction varies by no more than about 15%;
54. The injection/implant composition according to any one of Aspects 49-53, wherein the particles are microspheres, at least about 85% of which have an average maximum diameter of between 30 μm and 40 μm;
55. The injection/implant composition of any one of Aspects 49-54, wherein the composition is in the form of an aqueous liquid or a gel and the carrier comprises (a) one or more physiological acceptable surfactants and (b) one or more physiologically acceptable gelling agents;
56. The injection/implant composition of Aspect 55, wherein the excipient and diluent components of the liquid or gel have an average resorption time of less than about 4 months in humans;
57. The injection/implant composition of Aspect 56, wherein the excipient and diluent components of the liquid or gel has an average resorption time of about 2 months or less in humans;
58. The injection/implant composition of Aspect 56 or Aspect 57, wherein the liquid or gel further comprises one or more physiologically acceptable buffers, one or more physiologically acceptable salts, one or more physiologically acceptable preservatives, or a combination of two or more thereof;
59. The injection/implant composition of any one of Aspects 49-58, wherein for at least 97.5% of the particles in the composition the maximum size of an agglomeration of three injection/implant particles is less than 200 μm;
60. The injection/implant composition of any one of Aspects 49-59, wherein the presence and/or effect of the injection/implant particles is detectable in humans for an average of at least one year after administration;
61. The injection/implant composition of Aspect 60, wherein the presence and/or effect of the injection/implant particles is detectable in humans for an average of at least about 2 years after administration;
62. The injection/implant composition of any one of Aspects 49-61, wherein the injection/implant composition results in a detectable allergenic reaction in less than about 1% of human recipients;
63. The injection/implant composition of any one of Aspects 49-62, wherein the injection/implant composition can be classified as a non-pyrogenic composition and results in a detectable inflammatory reference in less than about 1% of human recipients;
64. A composition of between about 200 mg and about 500 mg of an injection/implant composition comprising an injection/implant composition according to any one of Aspects 1-63 and further comprising one or more excipients;
65. The composition of Aspect 64, wherein the composition comprises about 250 mg to about 450 mg of an injection/implant composition;
66. The composition of Aspect 65, wherein the composition comprises about 325 mg to about 425 mg of an injection/implant composition according to any one of Aspects 1-63;
67. The composition of Aspect 66, wherein the composition comprises about 350 mg to about 410 mg of an injection/implant composition according to any one of Aspects 1-56;
68. The composition of any one of Aspects 64-67 wherein the concentration of particles in the composition, when reconstituted with water for injection and hence prepared for injection, is about 10-about 30 mg/mL;
69. The composition of Aspect 68, wherein the concentration of particles in the composition is about 15-25 mg/mL;
70. The composition of Aspect 69, wherein the concentration of particles in the composition is about 20 mg/mL;
71. The composition of any one of Aspects 64-70, wherein the concentration of particles in the composition (without water or diluent) is about 25% to about 100%;
72. The composition of Aspect 71, wherein the concentration of the particles in the composition is about 50%-about 100%;
73. The composition of any one of Aspects 64-72, wherein the composition is a dry, solid composition;
74. The composition of Aspect 73, wherein the composition is a lyophilized composition;
75. The composition of any one of Aspects 64-72, wherein the composition is an aqueous liquid or gel composition;
76. The composition of Aspect 75, wherein the composition comprises about 3.5-about 10.5 mL water for injection;
77. The composition of Aspect 76, wherein the composition comprises about 4-about 9 mL water for injection;
78. The composition of Aspect 77, wherein the composition comprises about 4.5 mL to about 8.5 mL water for injection;
79. The composition of any one of Aspects 75-78 wherein the concentration of the formulation comprising the combination of the particles and the carrier in the aqueous liquid or gel composition is about 35-65 mg/mL;
80. The composition of Aspect 79, wherein the concentration of the formulation in the aqueous liquid or gel composition is about 40-60 mg/mL;
81. The composition of Aspect 80, wherein the concentration of the formulation in the aqueous liquid or gel composition is about 45-55 mg/mL;
82. The composition of Aspect 81, wherein the concentration of particles in the composition is about 1.5 wt. % to about 2.5 wt. %;
83. The composition of Aspect 82, wherein the concentration of particles in the composition is about 1.9 wt. % to about 2.1 wt. %;
84. The composition of Aspect 80, wherein the concentration of the formulation in the aqueous liquid or gel composition is about 3.5 wt. %-about 6 wt. %;
85. The composition of Aspect 80, wherein the concentration of the formulation in the aqueous liquid or gel composition is about 4.5 wt. %-about 5.3 wt. %;
86. An injection/implant delivery system comprising (a) a storage component containing an effective amount of an injection/implant composition according to any one of Aspects 1-78 and (b) a needle or cannula configured to deliver the composition to the mammal upon application of a delivery force;
87. The injection/implant delivery system of Aspect 86, wherein the system comprises a needle;
88. The injection/implant delivery system of Aspect 87, wherein the inner diameter of the needle is about 0.1 mm to about 0.4 mm;
89. The injection/implant delivery system of Aspect 88, wherein the inner diameter of the needle is about 0.125 mm to about 0.285 mm;
90. The injection/implant delivery system of Aspect 89, wherein the inner diameter of the needle is about 0.11 mm to about 0.35 mm;
91. The injection/implant delivery system of any one of Aspects 88-90, wherein the outer diameter of the needle is about 0.1 mm to about 0.7 mm;
92. The injection/implant delivery system of Aspect 91, wherein the outer diameter of the needle is about 0.23 to about 0.59 mm;
93. The injection/implant delivery system of Aspect 92, wherein the needle and/or cannula ranges in size between 24 Gauge (24G) and 31 Gauge (G);
94. The injection/implant delivery system of Aspect 93, wherein the needle is selected from the group consisting of a 26G needle, a 27G needle, or a 28G needle;
95. The injection/implant delivery system of Aspect 94, wherein the needle is a 26G needle;
96. The injection/implant delivery system of any one of Aspects 86-95, wherein the amount of amount of injection/implant composition in the delivery system is about 200 mg to about 500 mg;
97. The neocollagenic dermatologic implant delivery system of Aspect 96, wherein the about 200 mg to about 500 mg of implant composition is present in an amount of about 3.5 mg to about 10.5 mL of water for injection;
98. A kit or packaged product comprising (a) a container comprising a dried, solid composition according to any one of Aspects 1-74, (b) an aqueous solution suitable for reconstituting the composition to form an aqueous liquid or gel composition, and (c) a delivery system comprising a needle or a cannula configured to deliver the aqueous liquid or gel composition upon application of a delivery force into a mammalian recipient;
99. The kit according to Aspect 98, wherein the delivery system comprises a needle;
100. A method of delivering an injection/implant composition in the form of an aqueous liquid or gel composition to a mammalian recipient comprising injecting a mammalian recipient with the needle of a delivery system according to any one of Aspects 86-97 and applying a delivery force to the delivery system to deliver an effective amount of the injection/implant composition to the mammalian recipient;
101. The method of Aspect 100, wherein the mammalian recipient is a human;
102. The method of Aspect 101, wherein the human is a patient for aesthetic treatment under the supervision of a licensed healthcare provider;
103. The method of Aspect 102, wherein the risk of injection failure associated with use of the delivery system is about 40% or less;
104. The method of Aspect 103, wherein the risk of injection failure associated with the use of the delivery system is less than about 30%;
105. The method of Aspect 104, wherein the risk of injection failure associated with the use of the delivery system is less than about 15%;
106. The method of any one of Aspects 100-105, wherein the minimum force used to deliver the injection/implant composition is about 40 to about 80 g;
107. The method of Aspect 106, wherein the minimum force used to deliver the composition is about 40-55 g;
108. The method of Aspect 107, wherein the minimum force used to deliver the composition is less than about 50 g;
109. The method of any one of Aspects 100-108, wherein the maximum force used to deliver the composition is about 150 g-about 2500 g;
110. The method of Aspect 109, wherein the maximum force used to deliver the composition is about 175 g-1750 g;
111. The method of Aspect 110, wherein the maximum force used to deliver the composition is about 200 g-about 1200 g;
112. The method of Aspect 111, wherein the maximum force used to deliver the composition is about 200 g-about 700 g;
113. The method of any one of Aspects 99-112, wherein the mean force used to deliver the composition is about 100 g-1000 g;
114. The method of Aspect 113, wherein the mean force used to deliver the composition is about 150 g-750 g;
115. The method of Aspect 114, wherein the mean force used to deliver the composition is about 175 g-about 400 g;
116. The method of Aspect 115, wherein the mean force used to deliver the composition is about 200 g-about 350 g;
117. A method of increasing the volume of the skin in a mammalian recipient comprising administering an effective amount of a composition according to any one of Aspects 1-85 to the mammalian recipient;
118. The method of Aspect 110, wherein the method comprises performing the steps of the method of any one of Aspects 100-116;
119. The method of Aspect 117 or Aspect 118, wherein the method comprises repeat administration of the injection/implant composition on different days as part of a course of treatment;
120. The method of Aspect 119, wherein the course of treatment comprises performing 3-4 administrations of the injection/implant composition;
121. The method of any one of Aspects 117-120, wherein the composition comprises an anesthetic or the method is performed in association with the administration of an anesthetic;
122. The method of any one of Aspects 117-121, wherein the method is performed in combination with application of another aesthetic method;
123. The method of any one of Aspects 117-122, wherein the method comprises administering the composition to an area of human skin having an average epidermal thickness of less than 40.75 μm, an average dermal thickness of less than 1230 μm, or both;
124. The method of Aspect 123, wherein the method comprises administering the composition to an area of human skin having an average epidermal thickness of less than 40.5 μm, an average dermal thickness of less than 1200 μm, or both;
125. The method of Aspect 123 or Aspect 124, wherein the method comprises administering the composition to an area of human skin having an average epidermal thickness of less than 40 μm;
126. The method of any one of Aspects 123-125, wherein the method comprises administering the composition to an area of human skin having an average dermal thickness of less than 1175 μm;
127. The method of any one of Aspects 117-125, wherein the method comprises administering the composition to an area of human skin having an average relative thickness score of less than 1.5;
128. The method of Aspect 127, wherein the method comprises administering the composition to an area of human skin having an average relative thickness score of less than 1.35;
129. The method of any one of Aspects 117-128, wherein the method comprises administering the composition to an area of human skin in a part of the body, such as the face and neck, that has an average thickness that is at least 30% less than the thickness of the area of human skin in the part of the body that has the average thickest skin;
130. A composition according to any one of Aspects 1-80, wherein the mean particle size does not decrease by more than 10% over a course of time represented by or substantially equivalent to a 6 month accelerated stability study (wherein the composition is stored at about 40° C. and about 75% relative humidity), a long term ordinary condition stability study (wherein the composition is stored at about 25° C. and about 60% RH for 12 months), or both.
131. The composition of Aspect 131, wherein the polydispersity index of particles is stable over a course of time represented by or substantially equivalent to a 6 month accelerated stability study wherein the composition is stored at about 40° C. and about 75% relative humidity, such that the polydispersity index of particles does not vary by more than about 5%.
132. The composition of any one of Aspects 1-80, 130, and 131, wherein at least about 90% of the particles are microspheres and at least about 50% of the microspheres have a maximum diameter within a range of 30 μm+/−12.5 μm.
133. The composition of any one of Aspects 1-80 and 130-132, wherein the average maximum size of an agglomeration of three injectable particles is less than 200 μm.
134. The composition of any one of Aspects 1-80 and 130-133, wherein the average minimum force, maximum force, or both, required to deliver a dose of the composition through a needle is only about 50% or less of the corresponding force required to deliver a corresponding dose of Sculptra® through the needle.

1. An composition suitable for injection for increasing dermal volume comprising an effective amount of neocollagenic particles that are at least primarily composed of one or more resorbable polyester polymers selected from the group comprising polylactic-co-glycolic acid (PLGA), a PLA, or a mixture of two or more thereof, and that have a size distribution such that at least about 70% of the particles have a maximum particle diameter that is within about 30% of the mean particle diameter.

2. The composition of claim 1, wherein at least about 80% of the particles have a maximum particle diameter that is within 25% of the mean particle diameter.

3. The composition of claim 2, wherein at least about 90% of the particles are microspheres and at least about 50% of the microspheres have a maximum diameter within a range of 30 μm+/−12.5 μm.

4. The composition of claim 3, wherein at least about 70% of the particles of the composition have a maximum diameter that is between about 27.5 μm to about 62.5 μm.

5. The composition of claim 4, wherein the average maximum size of an agglomeration of three injectable particles is less than 200 μm.

6. The composition of claim 5, wherein at least 50% of the particles consist essentially of a PLA.

7. The composition of claim 6, wherein the PLA is poly-L-lactic acid (PLLA) (crystalline), poly-D-lactic acid (PDLA) (crystalline), poly-D,L-lactic acid (PDLLA) (amorphous), or a mixture thereof.

8. The composition of claim 7, wherein at least 50% of the polyester polymer content of the composition is composed of PLLA.

9. The composition of claim 1, wherein the presence, the effect, or both the presence and the effect of the injectable particles is detectable in humans for an average of at least one year after administration.

10. The composition of claim 9, wherein dermal injection and implantation of an effective amount of the composition results in a measurable increase in dermal volume for an average period of at least about 2 years.

11. The composition of claim 9, wherein the composition is in the form of an aqueous liquid or gel further comprising a physiological suitable carrier comprising one or more physiologically acceptable surfactants and one or more physiologically acceptable gelling agents, the particles having a concentration of 10 mg/mL-30 mg/mL.

12. The composition of aspect 11, wherein the composition can be delivered by injection through a needle having an inner diameter of between about 0.1 mm-about 0.4 mm needle with a failure rate of less than 40%.

13. The composition of claim 11, wherein the average minimum force, maximum force, or both, required to deliver a dose of the composition through a needle is only about 50% or less of the corresponding force required to deliver a corresponding dose of Sculptra® through the needle.

14. The composition of claim 8, wherein mean particle size does not decrease by more than 10% during a 6-month accelerated stability study wherein the composition is stored at 38-42° C. and about 70-80% relative humidity.

15. The composition of claim 14, wherein polydispersity index of the particles does not vary by more than 5% when the composition is stored at 38-42° C. and 70-80% relative humidity for a period of six months.

16. A method of increasing the volume of the skin by delivering by injection of between about 200 mg-about 500 mg of an aqueous liquid or gel dermal implant composition wherein the composition comprises a filler component that is composed of an effective amount of resorbable polyester particles that consist essentially of poly-L-lactic acid (PLLA) and a physiologically suitable carrier and, further, wherein the particles have a size distribution such that at least about 70% of the particles have a maximum particle diameter that is within about 30% of the mean particle diameter.

17. The method of claim 16, wherein at least about 90% of the particles are microspheres and at least about 50% of the microspheres have a maximum diameter within a range of 30 μm+/−12.5 μm.

18. The method of claim 17, wherein the dermal implant composition is capable of being delivered using a needle delivery system comprising a needle having an internal diameter of between about 0.1 mm-about 0.44 mm with a failure rate of the needle delivery system of less than 40%.

19. The method of claim 18, wherein the resorbable polyester particles are present in a final concentration of 10 mg/mL-30 mg/mL.

20. The method of claim 18, wherein the average minimum force, average maximum force, or both, required to deliver a dose of the composition through a needle is only about 50% or less of the corresponding force required to deliver a corresponding dose of SCULPTRA® through the needle.
